(19) United States
(12) Patent Application Publication
(10) Pub. No.: US 2026/0108251 A1
(43) Pub. Date: Apr. 23, 2026

(54) DEVICES, SYSTEMS, AND METHODS FOR OCCLUDING A LEFT ATRIAL APPENDAGE

(71) Applicant: BOSTON SCIENTIFIC SCIMED, INC., MAPLE GROVE, MN (US)

(72) Inventors: Philip Andrew Litecky, Forest Lake, MN (US); Troy Anthony Giese, Blaine, MN (US)

(73) Assignee: BOSTON SCIENTIFIC SCIMED, INC., MAPLE GROVE, MN (US)

(21) Appl. No.: 19/364,182

(22) Filed: Oct. 21, 2025

Related U.S. Application Data
(60) Provisional application No. 63/710,295, filed on Oct. 22, 2024.

(51) Int. Cl. A61B 17/12; A61B 17/00

(52) U.S. Cl. CPC A61B 17/12122; A61B 17/12163; A61B 17/12181; A61B 2017/00243; A61B 2017/00292; A61B 2017/12054

(57) ABSTRACT

A device for occluding a left atrial appendage includes an implant anchor element including a first locking structure, a first piece of foam including a second locking structure, and a second piece of foam including a third locking structure. A system includes a guide sheath, a first delivery device coupled to the implant anchor element, a second delivery device coupled to the first piece of foam, and a third delivery device coupled to the second piece of foam. A method of occluding the left atrial appendage includes advancing the implant anchor element to the appendage within the guide sheath, securing the implant anchor element to tissue, advancing the first piece of foam to the appendage, engaging the first locking structure with the second locking structure, advancing the second piece of foam to the appendage, and engaging the third locking structure with the second locking structure.

### CROSS-REFERENCE TO RELATED APPLICATIONS

This application claims the benefit of priority of U.S. Provisional Application No. 63/710,295 filed October 22, 2024, the entire disclosure of which is hereby incorporated by reference.

### TECHNICAL FIELD

The disclosure relates generally to medical devices and more particularly to medical devices, systems, and methods that are adapted for use in percutaneous medical procedures including implantation into the left atrial appendage (LAA) of a heart.

### BACKGROUND

The left atrial appendage is a small organ attached to the left atrium of the heart. During normal heart function, as the left atrium constricts and forces blood into the left ventricle, the left atrial appendage constricts and forces blood into the left atrium. The ability of the left atrial appendage to contract assists with improved filling of the left ventricle, thereby playing a role in maintaining cardiac output. However, in patients suffering from atrial fibrillation, the left atrial appendage may not properly contract or empty, causing stagnant blood to pool within its interior, which can lead to the undesirable formation of thrombi within the left atrial appendage.

Thrombi forming in the left atrial appendage may break loose from this area and enter the blood stream. Thrombi that migrate through the blood vessels may eventually plug a smaller vessel downstream and thereby contribute to stroke or heart attack. Clinical studies have shown that the majority of blood clots in patients with atrial fibrillation originate in the left atrial appendage. As a treatment, medical devices have been developed which are deployed to close off the left atrial appendage. Of the known medical devices, systems, and methods, each has certain advantages and disadvantages. There is an ongoing need to provide alternative medical devices and systems, and methods for manufacturing and using medical devices and systems.

### SUMMARY

In one example, a device for occluding a left atrial appendage may comprise an implant anchor element comprising a first locking structure fixedly attached to the implant anchor element, wherein the implant anchor element is securable to tissue within the left atrial appendage, a first piece of foam comprising a second locking structure fixedly attached to the first piece of foam, wherein the second locking structure is configured to engage the first locking structure to secure the first piece of foam to the implant anchor element, and a second piece of foam comprising a third locking structure fixedly attached to the second piece of foam, wherein the third locking structure is configured to engage the second locking structure to secure the second piece of foam to the first piece of foam.

In addition, or alternatively, to any example disclosed herein, the second locking structure is configured to passively lock to the first locking structure.

In addition, or alternatively, to any example disclosed herein, the third locking structure is configured to passively lock to the second locking structure.

In addition, or alternatively, to any example disclosed herein, the second locking structure is configured to actively lock to the first locking structure.

In addition, or alternatively, to any example disclosed herein, the third locking structure is configured to actively lock to the second locking structure.

In addition, or alternatively, to any example disclosed herein, a portion of the second locking structure is configured to extend into the first locking structure when the second locking structure is engaged with the first locking structure.

In addition, or alternatively, to any example disclosed herein, a portion of the third locking structure is configured to extend into the second locking structure when the third locking structure is engaged with the second locking structure.

In addition, or alternatively, to any example disclosed herein, the device may comprise a third piece of foam comprising a fourth locking structure fixedly attached to the third piece of foam, wherein the fourth locking structure is configured to engage the third locking structure to secure the third piece of foam to the second piece of foam.

In addition, or alternatively, to any example disclosed herein, the second locking structure is embedded within the first piece of foam.

In addition, or alternatively, to any example disclosed herein, the third locking structure is embedded within the second piece of foam.

In addition, or alternatively, to any example disclosed herein, and in another example, a system for occluding a left atrial appendage may comprise a guide sheath comprising a lumen, a first delivery device releasably coupled to an implant anchor element comprising a first locking structure fixedly attached to the implant anchor element, wherein the first delivery device is configured to slidably pass through the lumen of the guide sheath, a second delivery device releasably coupled to a first piece of foam comprising a second locking structure fixedly attached to the first piece of foam, wherein the second locking structure is configured to engage the first locking structure to secure the first piece of foam to the implant anchor element, wherein the second delivery device is configured to slidably pass through the lumen of the guide sheath, and a third delivery device releasably coupled to a second piece of foam comprising a third locking structure fixedly attached to the second piece of foam, wherein the third locking structure is configured to engage the second locking structure to secure the second piece of foam to the first piece of foam, wherein the third delivery device is configured to slidably pass through the lumen of the guide sheath.

In addition, or alternatively, to any example disclosed herein, the first delivery device is configured to engage the anchor implant element with tissue of the left atrial appendage.

In addition, or alternatively, to any example disclosed herein, the second delivery device is configured to translate the first piece of foam through the lumen of the guide sheath to engage the second locking structure with the first locking structure to secure the first piece of foam to the anchor implant element.

In addition, or alternatively, to any example disclosed herein, a first axial force applied to the second delivery device is required to translate the first piece of foam through the lumen of the guide sheath and a second axial force applied to the second delivery device is required to engage the second locking structure with the first locking structure, wherein the second axial force applied to the second delivery device is greater than the first axial force applied to the second delivery device.

In addition, or alternatively, to any example disclosed herein, the third delivery device is configured to translate the second piece of foam through the lumen of the guide sheath to engage the third locking structure with the second locking structure to secure the second piece of foam to the first piece of foam.

In addition, or alternatively, to any example disclosed herein, a first axial force applied to the third delivery device is required to translate the second piece of foam through the lumen of the guide sheath and a second axial force applied to the third delivery device is required to engage the third locking structure with the second locking structure, wherein the second axial force applied to the third delivery device is greater than the first axial force applied to the third delivery device.

In addition, or alternatively, to any example disclosed herein, a first portion of the second locking structure is movable relative to a second portion of the second locking structure to lock the second locking structure to the first locking structure.

In addition, or alternatively, to any example disclosed herein, a first portion of the third locking structure is movable relative to a second portion of the third locking structure to lock the third locking structure to the second locking structure.

In addition, or alternatively, to any example disclosed herein, and in another example, a method of occluding a left atrial appendage may comprise: advancing an implant anchor element releasably coupled to a first delivery device to the left atrial appendage within a lumen of a guide sheath; securing the implant anchor element to tissue within the left atrial appendage; removing the first delivery device from the lumen of the guide sheath; advancing a first piece of foam releasably coupled to a second delivery device to the left atrial appendage within the lumen of the guide sheath; engaging a first locking structure of the implant anchor element with a second locking structure of the first piece of foam to secure the first piece of foam to the implant anchor element within the left atrial appendage; removing the second delivery device from the lumen of the guide sheath; advancing a second piece of foam releasably coupled to a third delivery device to the left atrial appendage within the lumen of the guide sheath; and engaging a third locking structure of the second piece of foam with the second locking structure of the first piece of foam to secure the second piece of foam to the first piece of foam within the left atrial appendage.

In addition, or alternatively, to any example disclosed herein, engaging the first locking structure with the second locking structure comprises moving a first portion of the second locking structure relative to a second portion of the second locking structure to lock the second locking structure to the first locking structure; and engaging the second locking structure with the third locking structure comprises moving a first portion of the third locking structure relative to a second portion of the third locking structure to lock the third locking structure to the second locking structure.

The above summary of some embodiments, aspects, and/or examples is not intended to describe each embodiment or every implementation of the present disclosure. The figures and the detailed description more particularly exemplify aspects of these embodiments.

### BRIEF DESCRIPTION OF THE DRAWINGS

The disclosure may be more completely understood in consideration of the following detailed description in connection with the accompanying drawings, in which:

FIG. 1 schematically illustrates selected aspects of a device and/or a system being deployed within a left atrial appendage;

FIG. 2A schematically illustrates selected aspects of an implant anchor element of the device and/or system of FIG. 1;

FIG. 2B schematically illustrates selected aspects of an alternative configuration of the implant anchor element of FIG. 2A;

FIG. 3 schematically illustrates selected aspects of an implant anchor element of the device and/or system of FIG. 1;

FIG. 4 schematically illustrates selected aspects of an implant anchor element of the device and/or system of FIG. 1;

FIGS. 5-7 schematically illustrate selected aspects of the device and/or the system of FIG. 1 being deployed within a left atrial appendage;

FIGS. 8-9 schematically illustrate selected aspects of locking structures of the device and/or system of FIG. 1;

FIG. 10 schematically illustrates selected aspects of locking structures of the device and/or system of FIGS. 8-9;

FIG. 11 is a cross-sectional view taken along the line 11-11 of FIG. 10;

FIGS. 12-13 schematically illustrate selected aspects of the locking structures of FIG. 10;

FIG. 14 schematically illustrates selected aspects of locking structures of the device and/or system of FIG. 1;

FIGS. 15-18 schematically illustrate selected aspects of locking structures of the device and/or system of FIG. 14;

FIGS. 19-20 schematically illustrate selected aspects of locking structures of the device and/or system of FIG. 1; and

FIGS. 21-24 schematically illustrate selected aspects of locking structures of the device and/or system of FIGS. 19-20.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently – such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

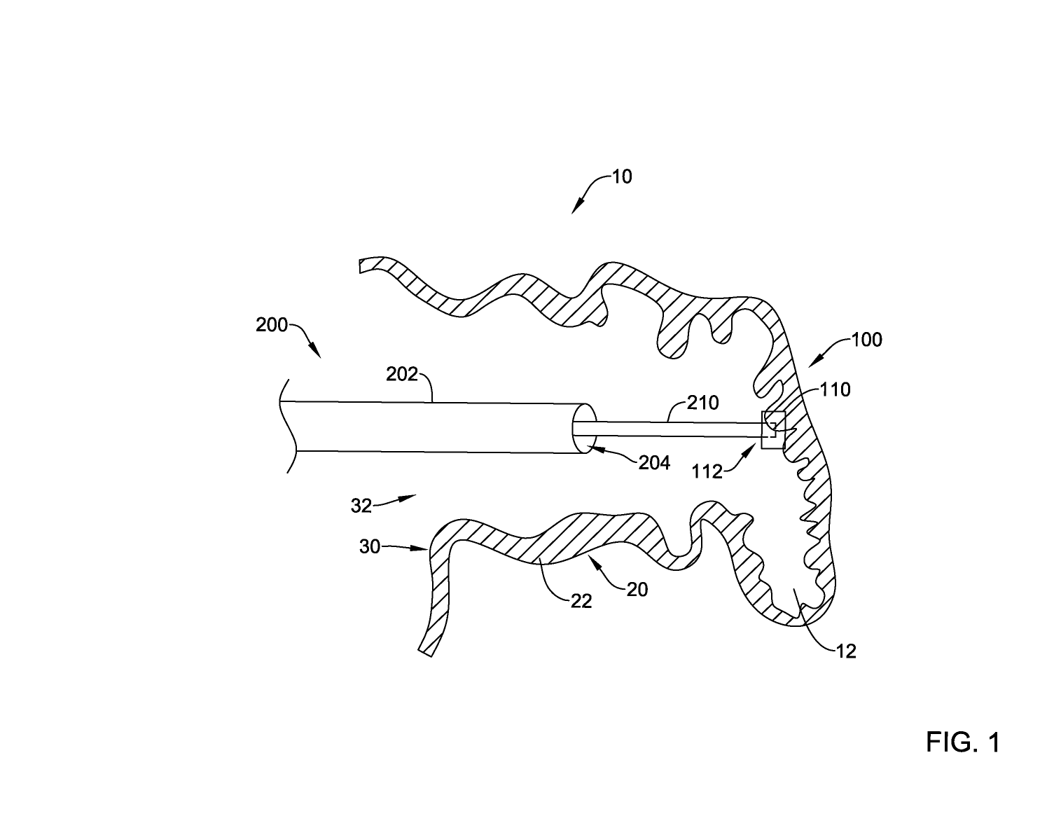

FIG. 1 schematically illustrates selected aspects of a left atrial appendage 10. It shall be understood that the left atrial appendage 10 of FIG. 1 is merely exemplary, and other geometries, shapes, sizes, etc. for the left atrial appendage 10 are common and may vary from patient to patient. The left atrial appendage 10 may be formed as a small pouch or extension attached to and extending from the left atrium of a patient’s heart.

The left atrial appendage 10 may include a longitudinal axis arranged along a depth of a main body 20 of the left atrial appendage 10. The main body 20 may include a side wall 22 and an ostium 30 forming a proximal mouth 32. In some embodiments, a lateral extent of the main body 20 and/or the ostium 30 may be smaller or less than a depth of the main body 20 along the longitudinal axis, or a depth of the main body 20 may be greater than a lateral extent of the main body 20 and/or the ostium 30. In some embodiments, the left atrial appendage 10 may narrow quickly along the depth of the main body 20 or the left atrial appendage 10 may maintain a generally constant lateral extent along a majority of the depth of the main body 20.

In some embodiments, the left atrial appendage 10 may include a distalmost region 12 formed or arranged as a tail-like element associated with a distal portion of the main body 20. In some embodiments, the distalmost region 12 may protrude radially or laterally away from the longitudinal axis and/or the main body 20.

FIG. 1 schematically illustrates selected aspects of a device 100 for occluding the left atrial appendage 10 and/or a system 200 for occluding the left atrial appendage 10. In at least some embodiments, the system 200 may include the device 100. In some embodiments, the system 200 may comprise a guide sheath 202 comprising a lumen 204 extending therein and/or therethrough. The guide sheath 202 may be configured to be advanced and/or navigated to the left atrial appendage 10. In one non-limiting example, the guide sheath 202 may be configured to be advanced and/or navigated percutaneously to the left atrial appendage 10 (e.g., through a patient’s vasculature, etc.). Other configurations are also contemplated. In some embodiments, the guide sheath 202 may be configured to be advanced and/or navigated over and/or along a guidewire to the left atrial appendage 10. Other configurations are also contemplated.

In some embodiments, the device 100 may comprise an implant anchor element 110 comprising a first locking structure 112 fixedly attached to the implant anchor element 110. In some embodiments, the first locking structure 112 may be monolithically formed with the implant anchor element 110. Other configurations are also contemplated.

The implant anchor element 110 may be securable to tissue within the left atrial appendage 10 (e.g., the side wall 22 of the left atrial appendage 10). For the purpose of illustration only, the implant anchor element 110 is shown in FIG. 1 as a “black box”. The implant anchor element 110 may take different forms, as discussed herein, which may be used in place of and/or may be assumed to take the place of the “black box” in FIG. 1.

In some embodiments, the system 200 may comprise a first delivery device 210 releasably coupled to the implant anchor element 110 and/or the first locking structure 112. The first delivery device 210 may be configured to slidably pass through the lumen 204 of the guide sheath 202. In some embodiments, the first delivery device 210 may comprise an elongate shaft configured to releasably couple with the implant anchor element 110 and/or the first locking structure 112. In some embodiments, the first delivery device 210 may comprise a first delivery catheter having a lumen extending therethrough. In some embodiments, the elongate shaft of the first delivery device 210 may be slidably disposed within the lumen of the first delivery catheter. Other configurations are also contemplated.

The first delivery device 210 and/or the elongate shaft of the first delivery device 210 may be configured to engage the anchor implant element 110 with tissue of the left atrial appendage 10, as shown in FIG. 1. Thereafter, the first delivery device 210 and/or the elongate shaft of the first delivery device 210 may be disengaged from the anchor implant element 110. In some embodiments, the first delivery device 210 may be releasably coupled to the implant anchor element 110 and/or the first locking structure 112 via a threaded connection. In some embodiments, the first delivery device 210 may be releasably coupled to the implant anchor element 110 and/or the first locking structure 112 via a snap-fit mechanism. In some embodiments, the first delivery device 210 may be releasably coupled to the implant anchor element 110 and/or the first locking structure 112 via a spring-loaded mechanism. In some embodiments, the first delivery device 210 may be releasably coupled to the implant anchor element 110 and/or the first locking structure 112 via a friction-fit mechanism. Other configurations are also contemplated.

In some embodiments, a method of occluding the left atrial appendage 10 may comprise advancing the implant anchor element 110 releasably coupled to the first delivery device 210 to the left atrial appendage 10 within the lumen 204 of the guide sheath 202. In some embodiments, the method may comprise advancing the implant anchor element 110 to the left atrial appendage 10 within the first delivery catheter. In some embodiments, the guide sheath 202 may be advanced to the left atrial appendage 10 over and/or along a guidewire. Thereafter, the first delivery device 210 may be advanced through the guide sheath 202 to the left atrial appendage 10. In at least some embodiments, the guidewire may be removed prior to advancing the first delivery device 210 through the lumen 204 of the guide sheath 202. Other configurations are also contemplated. In some embodiments, the method of occluding the left atrial appendage 10 may comprise securing the implant anchor element 110 to tissue within the left atrial appendage 10.

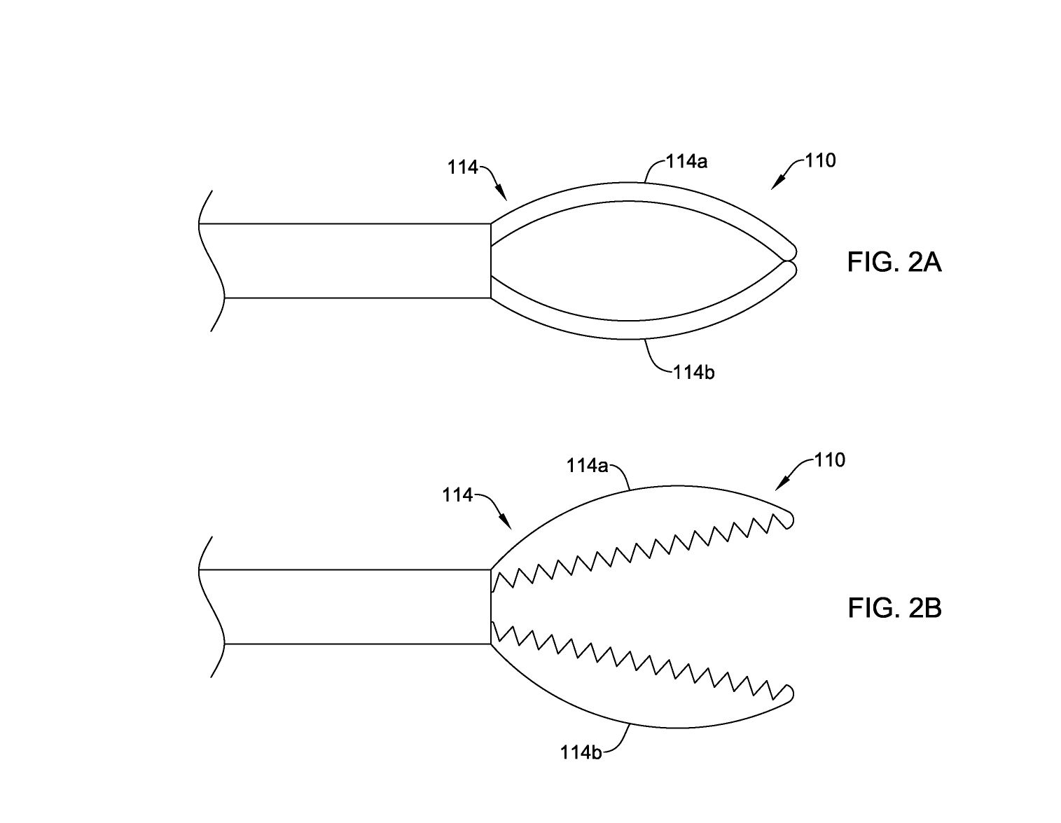

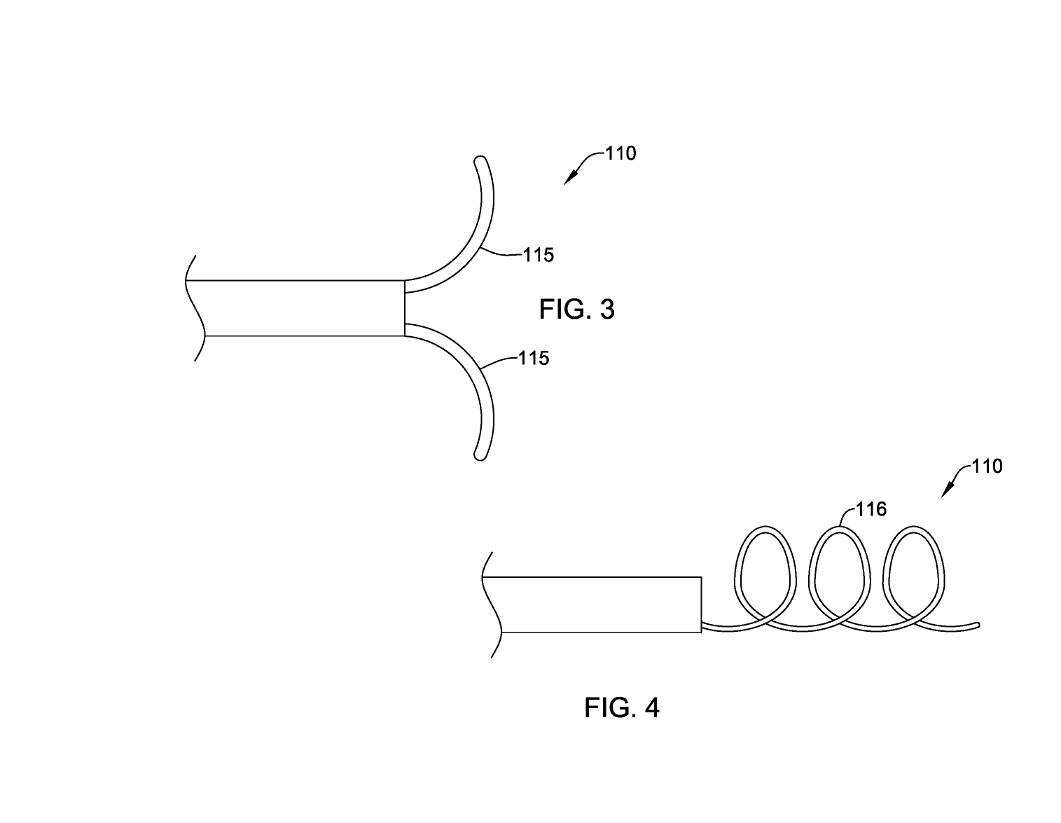

FIG. 2A, FIG. 2B, FIG. 3, and FIG. 4 schematically illustrate selected aspects of and alternative configurations of an implant anchor element of the device and/or system of FIG. 1. In some embodiments, the implant anchor element 110 may comprise a grasping structure 114 configured to pinch and/or grasp tissue of the left atrial appendage 10. In some embodiments, the grasping structure 114 may be actuatable via an actuation mechanism. In some embodiments, the grasping structure 114 may be shape memory biased toward a closed configuration. Other configurations are also contemplated. In some embodiments, the grasping structure 114 may comprise a first jaw 114A and a second jaw 114B hingedly and/or pivotably coupled together. In some embodiments, the first jaw 114A and the second jaw 114B may be arcuate elements, as seen in FIG. 2A, configured to pinch and/or grasp tissue of the left atrial appendage 10 between distal tips thereof. In some embodiments, the first jaw 114A and/or the second jaw 114B may comprise teeth, as seen in FIG. 2B, configured to pinch and/or grasp tissue of the left atrial appendage 10 between the first jaw 114A and/or the second jaw 114B. Other configurations are also contemplated.

In some embodiments, the implant anchor element 110 may comprise one or more hooks 115, as seen in FIG. 3, configured to engage and/or penetrate tissue of the left atrial appendage 10. In some embodiments, the one or more hooks 115 may be configured to engage with a surface of the tissue of the left atrial appendage 10. In some embodiments, the one or more hooks 115 may be configured to extend and/or penetrate under the surface of the tissue of the left atrial appendage 10. Other configurations are also contemplated. In some embodiments, the implant anchor element 110 may comprise a helical structure 116, as seen in FIG. 4, configured to engage and/or penetrate tissue of the left atrial appendage 10. In some embodiments, the helical structure 116 may be configured to screw into tissue of the left atrial appendage 10. Other configurations are also contemplated.

After securing the implant anchor element 110 to tissue within the left atrial appendage, the first delivery device 210 may be decoupled from the implant anchor element 110 and/or the first locking structure 112. The method of occluding the left atrial appendage 10 may comprise subsequently removing the first delivery device 210 from the lumen 204 of the guide sheath 202.

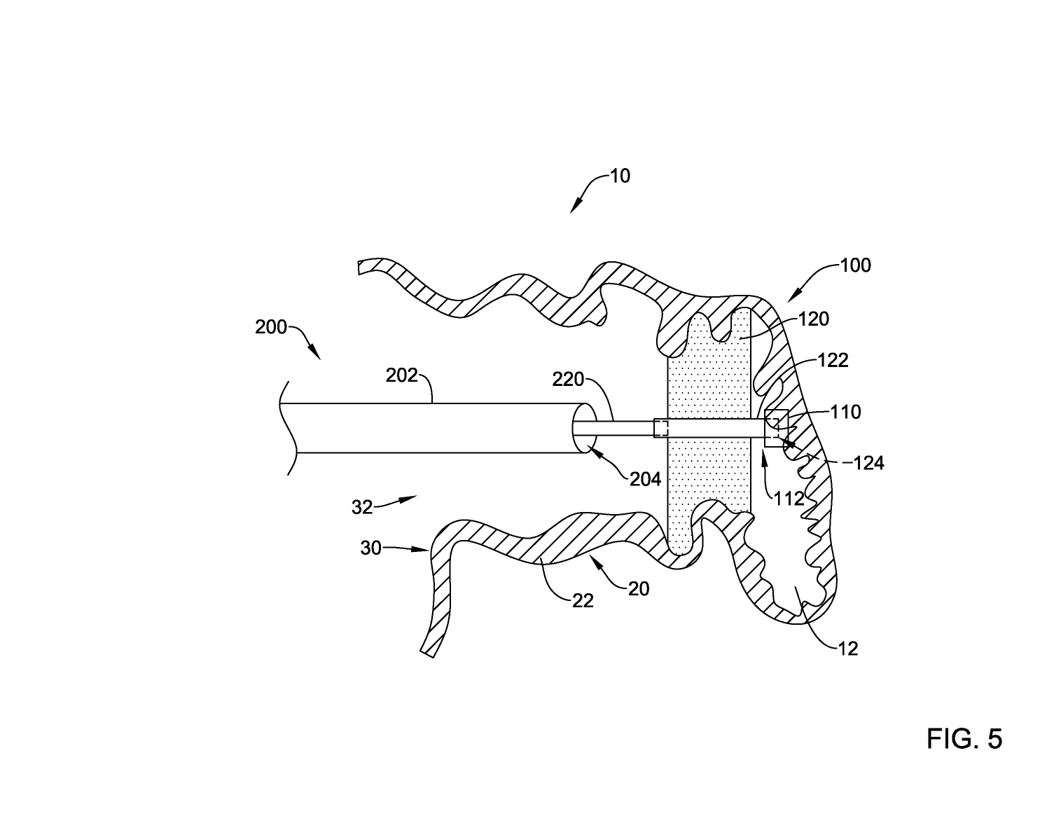

FIG. 5 schematically illustrates selected aspects of the device and/or the system of FIG. 1 being deployed within the left atrial appendage 10. In some embodiments, the device 100 may comprise a first piece of foam 120 comprising a second locking structure 122 fixedly attached to the first piece of foam 120, as seen in FIG. 5. In some embodiments, the second locking structure 122 may be embedded within the first piece of foam 120. In some embodiments, the second locking structure 122 may be adhesively bonded to the first piece of foam 120. Other configurations are also contemplated. In some embodiments, the second locking structure 122 may be configured to engage the first locking structure 112 to secure the first piece of foam 120 to the implant anchor element 110. In some embodiments, the second locking structure 122 may be configured to passively lock to and/or with the first locking structure 112. In some embodiments, the second locking structure 122 may be configured to actively lock to and/or with the first locking structure 112. For the purpose of this disclosure, a passive lock requires no additional effort and/or actuation by the user after the locking structures are engaged with each other such that the locking structures may be disengaged and/or detached from each other with sufficient effort and/or force but without breaking and/or physically damaging the locking structure(s). For the purpose of this disclosure, an active lock requires an extra step and/or action by the user to actuate some feature of the locking structure(s) to lock them together such that the locking structures cannot be disengaged and/or detached from each other without breaking and/or physically damaging the locking structure(s).

In some embodiments, a portion 124 of the second locking structure 122 may be configured to extend into the first locking structure 112 when the second locking structure 122 is engaged with the first locking structure 112. In some alternative embodiments, a portion of the second locking structure 122 may be configured to extend over and/or around the first locking structure 112 when the second locking structure 122 is engaged with the first locking structure 112. Other configurations are also contemplated.

In some embodiments, the system 200 may comprise a second delivery device 220 releasably coupled to the second locking structure 122. The second delivery device 220 may be configured to slidably pass through the lumen 204 of the guide sheath 202. In some embodiments, the second delivery device 220 may comprise an elongate shaft configured to releasably couple with the second locking structure 122. In some embodiments, the second delivery device 220 may comprise a second delivery catheter having a lumen extending therethrough. In some embodiments, the elongate shaft of the second delivery device 220 may be slidably disposed within the lumen of the second delivery catheter. Other configurations are also contemplated.

In some embodiments, the method of occluding the left atrial appendage 10 may comprise advancing the first piece of foam 120 releasably coupled to the second delivery device 220 to the left atrial appendage 10 within the lumen 204 of the guide sheath 202. The method of occluding the left atrial appendage 10 may comprise engaging the first locking structure 112 of the implant anchor element 110 with the second locking structure 122 of the first piece of foam 120 to secure the first piece of foam 120 to the implant anchor element 112 within the left atrial appendage 10, as seen in FIG. 5.

In some embodiments, engaging the first locking structure 112 of the implant anchor element 110 with the second locking structure 122 of the first piece of foam 120 may comprise axially moving a first portion of the second locking structure 122 relative to a second portion of the second locking structure 122 to lock the second locking structure 122 to the first locking structure 112, as discussed below.

The second delivery device 220 and/or the elongate shaft of the second delivery device 220 may be configured to engage the second locking structure 122 with the first locking structure 112. The second delivery device 220 and/or the elongate shaft of the second delivery device 220 may be configured to translate the first piece of foam 120 through the lumen 204 of the guide sheath 202 to engage the second locking structure 122 with the first locking structure 112 to secure the first piece of foam 120 to the anchor implant element 110. Thereafter, the second delivery device 220 and/or the elongate shaft of the second delivery device 220 may be disengaged from the second locking structure 122.

In some embodiments, the second delivery device 220 may be releasably coupled to the second locking structure 122 via a threaded connection. In some embodiments, the second locking structure 122 may be engaged with the first locking structure 112 via a threaded connection. In some embodiments, the second delivery device 220 may be releasably coupled to the second locking structure 122 via a snap-fit mechanism. In some embodiments, the second locking structure 122 may be engaged with the first locking structure 112 via a snap-fit mechanism. In some embodiments, the second delivery device 220 may be releasably coupled to the second locking structure 122 via a spring-loaded mechanism. In some embodiments, the second locking structure 122 may be engaged with the first locking structure 112 via a spring-loaded mechanism. In some embodiments, the second delivery device 220 may be releasably coupled to the second locking structure 122 via a friction-fit mechanism. In some embodiments, the second locking structure 122 may be engaged with the first locking structure 112 via a friction-fit mechanism. Other configurations are also contemplated.

After engaging the first locking structure 112 of the implant anchor element 110 with the second locking structure 122 of the first piece of foam 120 within the left atrial appendage, the second delivery device 220 may be decoupled from the second locking structure 122. The method of occluding the left atrial appendage 10 may comprise subsequently removing the second delivery device 220 from the lumen 204 of the guide sheath 202.

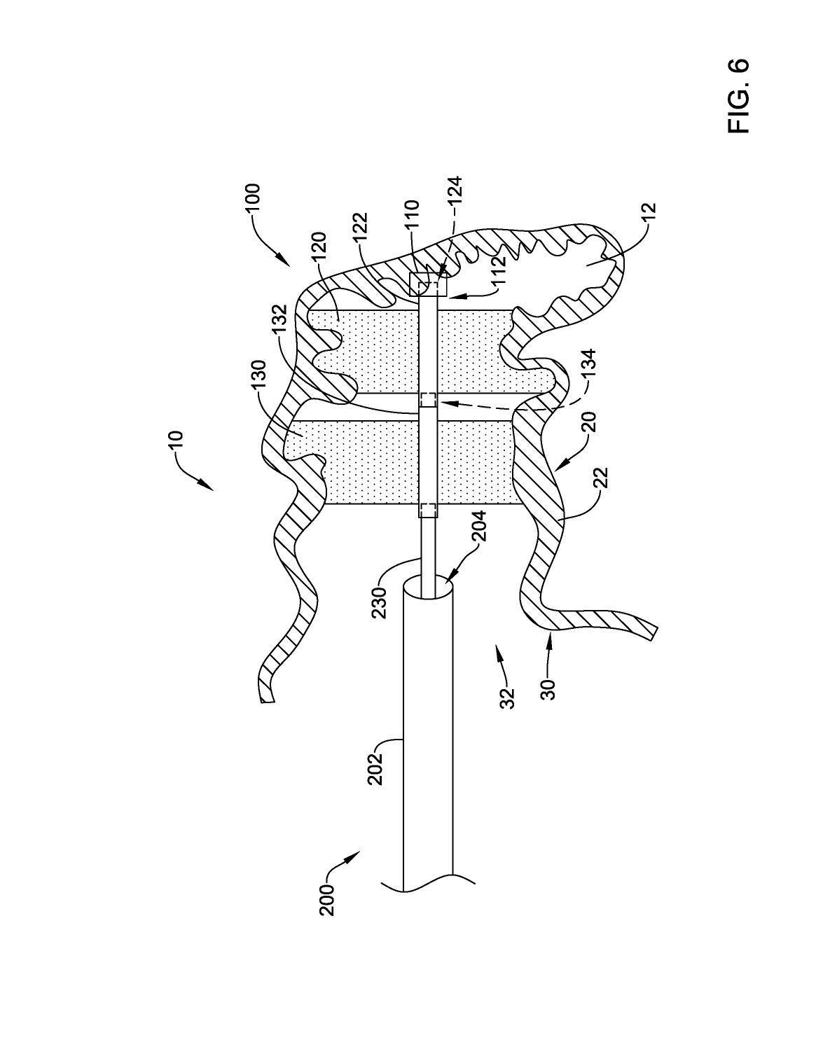

FIG. 6 schematically illustrates selected aspects of the device and/or the system of FIG. 1 being deployed within the left atrial appendage 10. In some embodiments, the device 100 may comprise a second piece of foam 130 comprising a third locking structure 132 fixedly attached to the second piece of foam 130, as seen in FIG. 6. In some embodiments, the third locking structure 132 may be embedded within the second piece of foam 130. In some embodiments, the third locking structure 132 may be adhesively bonded to the second piece of foam 130. Other configurations are also contemplated. In some embodiments, the third locking structure 132 may be configured to engage the second locking structure 122 to secure the second piece of foam 130 to the first piece of foam 120. In some embodiments, the third locking structure 132 may be configured to passively lock to and/or with the second locking structure 122. In some embodiments, the third locking structure 132 may be configured to actively lock to and/or with the second locking structure 122. In some embodiments, a portion 134 of the third locking structure 132 may be configured to extend into the second locking structure 122 when the third locking structure 132 is engaged with the second locking structure 122. In some alternative embodiments, a portion of the third locking structure 132 may be configured to extend over and/or around the second locking structure 122 when the third locking structure 132 is engaged with the second locking structure 122. Other configurations are also contemplated.

In some embodiments, the system 200 may comprise a third delivery device 230 releasably coupled to the third locking structure 132. The third delivery device 230 may be configured to slidably pass through the lumen 204 of the guide sheath 202. In some embodiments, the third delivery device 230 may comprise an elongate shaft configured to releasably couple with the third locking structure 132. In some embodiments, the third delivery device 230 may comprise a third delivery catheter having a lumen extending therethrough. In some embodiments, the elongate shaft of the third delivery device 230 may be slidably disposed within the lumen of the third delivery catheter. Other configurations are also contemplated.

In some embodiments, the method of occluding the left atrial appendage 10 may comprise advancing the second piece of foam 130 releasably coupled to the third delivery device 230 to the left atrial appendage 10 within the lumen 204 of the guide sheath 202. The method of occluding the left atrial appendage 10 may comprise engaging the third locking structure 132 of the second piece of foam 130 with the second locking structure 122 of the first piece of foam 120 to secure the second piece of foam 130 to the first piece of foam 120 within the left atrial appendage 10, as seen in FIG. 6.

In some embodiments, engaging the second locking structure 122 of the first piece of foam 120 with the third locking structure 132 of the second piece of foam 130 may comprise axially moving a first portion of the third locking structure 132 relative to a second portion of the third locking structure 132 to lock the third locking structure 132 to the second locking structure 122, as discussed below.

The third delivery device 230 and/or the elongate shaft of the third delivery device 230 may be configured to engage the third locking structure 132 with the second locking structure 122. The third delivery device 230 and/or the elongate shaft of the third delivery device 230 may be configured to translate the second piece of foam 130 through the lumen 204 of the guide sheath 202 to engage the third locking structure 132 with the second locking structure 122 to secure the second piece of foam 130 to the first piece of foam 120. Thereafter, the third delivery device 230 and/or the elongate shaft of the third delivery device 230 may be disengaged from the third locking structure 132.

In some embodiments, the third delivery device 230 may be releasably coupled to the third locking structure 132 via a threaded connection. In some embodiments, the third locking structure 132 may be engaged with the second locking structure 122 via a threaded connection. In some embodiments, the third delivery device 230 may be releasably coupled to the third locking structure 132 via a snap-fit mechanism. In some embodiments, the third locking structure 132 may be engaged with the second locking structure 122 via a snap-fit mechanism. In some embodiments, the third delivery device 230 may be releasably coupled to the third locking structure 132 via a spring-loaded mechanism. In some embodiments, the third locking structure 132 may be engaged with the second locking structure 122 via a spring-loaded mechanism. In some embodiments, the third delivery device 230 may be releasably coupled to the third locking structure 132 via a friction-fit mechanism. In some embodiments, the third locking structure 132 may be engaged with the second locking structure 122 via a friction-fit mechanism. Other configurations are also contemplated.

After engaging the third locking structure 132 of the second piece of foam 120 with the second locking structure 122 of the first piece of foam 120 within the left atrial appendage, the third delivery device 230 may be decoupled from the third locking structure 132. The method of occluding the left atrial appendage 10 may comprise subsequently removing the third delivery device 230 from the lumen 204 of the guide sheath 202.

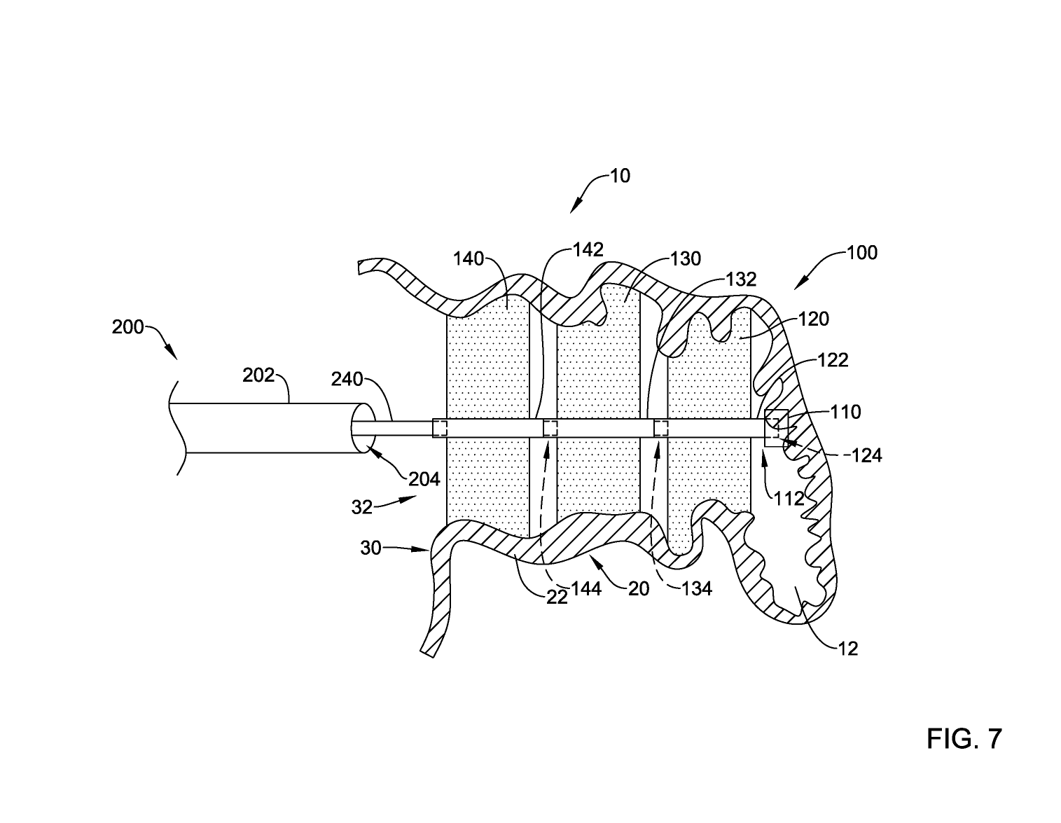

FIG. 7 schematically illustrates selected aspects of the device and/or the system of FIG. 1 being deployed within the left atrial appendage 10. In some embodiments, the device 100 may comprise a third piece of foam 140 comprising a fourth locking structure 142 fixedly attached to the third piece of foam 140, as seen in FIG. 7. In some embodiments, the fourth locking structure 142 may be embedded within the third piece of foam 140. In some embodiments, the fourth locking structure 142 may be adhesively bonded to the third piece of foam 140. Other configurations are also contemplated. In some embodiments, the fourth locking structure 142 may be configured to engage the third locking structure 132 to secure the third piece of foam 140 to the second piece of foam 130. In some embodiments, the fourth locking structure 142 may be configured to passively lock to and/or with the third locking structure 132. In some embodiments, the fourth locking structure 142 may be configured to actively lock to and/or with the third locking structure 132. In some embodiments, a portion 144 of the fourth locking structure 142 may be configured to extend into the third locking structure 132 when the fourth locking structure 142 is engaged with the third locking structure 132. In some alternative embodiments, a portion of the fourth locking structure 142 may be configured to extend over and/or around the third locking structure 132 when the fourth locking structure 142 is engaged with the third locking structure 132. Other configurations are also contemplated.

In some embodiments, the system 200 may comprise a fourth delivery device 240 releasably coupled to the fourth locking structure 142. The fourth delivery device 240 may be configured to slidably pass through the lumen 204 of the guide sheath 202. In some embodiments, the fourth delivery device 240 may comprise an elongate shaft configured to releasably couple with the fourth locking structure 142. In some embodiments, the fourth delivery device 240 may comprise a fourth delivery catheter having a lumen extending therethrough. In some embodiments, the elongate shaft of the fourth delivery device 240 may be slidably disposed within the lumen of the fourth delivery catheter. Other configurations are also contemplated.

In some embodiments, the method of occluding the left atrial appendage 10 may comprise advancing the third piece of foam 140 releasably coupled to the fourth delivery device 240 to the left atrial appendage 10 within the lumen 204 of the guide sheath 202. The method of occluding the left atrial appendage 10 may comprise engaging the fourth locking structure 142 of the third piece of foam 140 with the third locking structure 132 of the second piece of foam 130 to secure the third piece of foam 140 to the second piece of foam 130 within the left atrial appendage 10, as seen in FIG. 7.

In some embodiments, engaging the third locking structure 132 of the second piece of foam 130 with the fourth locking structure 142 of the third piece of foam 140 may comprise axially moving a first portion of the fourth locking structure 142 relative to a second portion of the fourth locking structure 142 to lock the fourth locking structure 142 to the third locking structure 132.

The fourth delivery device 240 and/or the elongate shaft of the fourth delivery device 240 may be configured to engage the fourth locking structure 142 with the third locking structure 132. The fourth delivery device 240 and/or the elongate shaft of the fourth delivery device 240 may be configured to translate the third piece of foam 140 through the lumen 204 of the guide sheath 202 to engage the fourth locking structure 142 with the third locking structure 132 to secure the third piece of foam 140 to the second piece of foam 130. Thereafter, the fourth delivery device 240 and/or the elongate shaft of the fourth delivery device 240 may be disengaged from the fourth locking structure 142.

In some embodiments, the fourth delivery device 240 may be releasably coupled to the fourth locking structure 142 via a threaded connection. In some embodiments, the fourth locking structure 142 may be engaged with the third locking structure 132 via a threaded connection. In some embodiments, the fourth delivery device 240 may be releasably coupled to the fourth locking structure 142 via a snap-fit mechanism. In some embodiments, the fourth locking structure 142 may be engaged with the third locking structure 132 via a snap-fit mechanism. In some embodiments, the fourth delivery device 240 may be releasably coupled to the fourth locking structure 142 via a spring-loaded mechanism. In some embodiments, the fourth locking structure 142 may be engaged with the third locking structure 132 via a spring-loaded mechanism. In some embodiments, the fourth delivery device 240 may be releasably coupled to the fourth locking structure 142 via a friction-fit mechanism. In some embodiments, the fourth locking structure 142 may be engaged with the third locking structure 132 via a friction-fit mechanism. Other configurations are also contemplated.

After engaging the fourth locking structure 142 of the third piece of foam 130 with the third locking structure 132 of the second piece of foam 130 within the left atrial appendage, the fourth delivery device 240 may be decoupled from the fourth locking structure 142. The method of occluding the left atrial appendage 10 may comprise subsequently removing the fourth delivery device 240 from the lumen 204 of the guide sheath 202.

The first piece of foam 120, the second piece of foam 130, the third piece of foam 140, etc. may be configured to shift from a first configuration (e.g., a compressed configuration) to a second configuration (e.g., an expanded configuration). In some embodiments, the first piece of foam 120, the second piece of foam 130, the third piece of foam 140, etc. may be constrained in the first configuration (e.g., the compressed configuration) by the second delivery device 220, the third delivery device 230, the fourth deliver device 240, etc., respectively. In some embodiments, the first piece of foam 120, the second piece of foam 130, the third piece of foam 140, etc. may be configured to shift from the first configuration (e.g., the compressed configuration) to the second configuration (e.g., the expanded configuration) when unconstrained.

In some embodiments, the first piece of foam 120, the second piece of foam 130, the third piece of foam 140, etc. may be configured to shift from the first configuration (e.g., the compressed configuration) to the second configuration (e.g., the expanded configuration) in vivo. In some embodiments, each of the first piece of foam 120, the second piece of foam 130, the third piece of foam 140, etc. may be configured to adapt and conform to the left atrial appendage 10, the side wall 22, and/or surrounding anatomy when shifting from the first configuration (e.g., the collapsed configuration) toward and/or to the second configuration (e.g., the expanded configuration) in vivo.

In some embodiments, the first piece of foam 120, the second piece of foam 130, the third piece of foam 140, etc. may be configured to shift from the first configuration (e.g., the compressed configuration) to the second configuration (e.g., the expanded configuration) when exposed to a stimulus, such as a preselected temperature, a fluid, a combination thereof, etc. In some embodiments, the first piece of foam 120, the second piece of foam 130, the third piece of foam 140, etc. may be formed from a shape memory material. In some embodiments, each of the first piece of foam 120, the second piece of foam 130, the third piece of foam 140, etc. may be formed from a different shape memory material. Other configurations are also contemplated.

In some embodiments, each of the first piece of foam 120, the second piece of foam 130, the third piece of foam 140, etc. may have a similar size (e.g., outer extent, axial thickness, etc.) in the first configuration (e.g., the compressed configuration). In some embodiments, each of the first piece of foam 120, the second piece of foam 130, the third piece of foam 140, etc. may have a different size (e.g., outer extent, axial thickness, etc.) in the second configuration (e.g., the expanded configuration). Other configurations are also contemplated.

In some embodiments, each of the first piece of foam 120, the second piece of foam 130, the third piece of foam 140, etc. may have a similar shape in the first configuration (e.g., the compressed configuration). In some embodiments, each of the first piece of foam 120, the second piece of foam 130, the third piece of foam 140, etc. may have a different shape in the second configuration (e.g., the expanded configuration). Other configurations are also contemplated.

In some embodiments, each of the first piece of foam 120, the second piece of foam 130, the third piece of foam 140, etc. may have a similar volume in the first configuration (e.g., the compressed configuration). In some embodiments, each of the first piece of foam 120, the second piece of foam 130, the third piece of foam 140, etc. may have a different volume in the second configuration (e.g., the expanded configuration). Other configurations are also contemplated.

In some embodiments, each of the first piece of foam 120, the second piece of foam 130, the third piece of foam 140, etc. may have a first overall volume in the first configuration (e.g., the compressed configuration). In some embodiments, each of the first piece of foam 120, the second piece of foam 130, the third piece of foam 140, etc. may have a second overall volume in the second configuration (e.g., the expanded configuration) different from the first overall volume. In some embodiments, the second overall volume may be about 10% different, about 20% different, about 30% different, about 40% different, about 50% different, about 75% different, about 100% different, about 125% different, about 150% different, about 200% different, etc. from the first overall volume. In at least some embodiments, the second overall volume may be greater than the first overall volume. In some alternative embodiments, the second overall volume may be less than the first overall volume.

In some embodiments, each of the first piece of foam 120, the second piece of foam 130, the third piece of foam 140, etc. may comprise and/or may be formed from a shape memory polymer and/or a shape memory foam. The shape memory polymer and/or the shape memory foam may have multiple geometric and/or mechanical properties when exposed to temperature, moisture, and/or chemical environments, and/or changes therein. In some embodiments, the shape memory polymer and/or the shape memory foam may have a collapsibility ratio that is high. The collapsibility ratio is a ratio between an expanded size and a collapsed or delivery size. In some examples, the collapsibility ratio of the shape memory polymer and/or the shape memory foam may be at least 5 times, at least 7 times, at least 8 times, at least 9 times, at least 10 times, at least 12 times, or more. In one non-limiting example, a piece of expandable foam may have an outer diameter of about 32 millimeters (1.260 inches) in the expanded configuration and about 3 millimeters (0.118 inches) in the compressed configuration, producing a collapsibility ratio of at least 10 times (e.g., at least 10:1). Other configurations are also contemplated. In at least some embodiments, each of the first piece of foam 120, the second piece of foam 130, the third piece of foam 140, etc. may be configured as open celled foam.

In some embodiments, each of the first piece of foam 120, the second piece of foam 130, the third piece of foam 140, etc. and/or the shape memory foam may be formed from a biocompatible material. In at least some embodiments, each of the first piece of foam 120, the second piece of foam 130, the third piece of foam 140, etc. may be non-biodegradable and/or non-bioabsorbable. In some alternative embodiments, each of the first piece of foam 120, the second piece of foam 130, the third piece of foam 140, etc. may be biodegradable and/or bioabsorbable over time. In some embodiments, each of the first piece of foam 120, the second piece of foam 130, the third piece of foam 140, etc. may be configured to promote endothelization and/or tissue ingrowth. In some embodiments, each of the first piece of foam 120, the second piece of foam 130, the third piece of foam 140, etc. may include a coating, a material, and/or a component that promotes endothelization and/or tissue ingrowth. Other configurations are also contemplated.

In some embodiments, each of the first piece of foam 120, the second piece of foam 130, the third piece of foam 140, etc. may be configured to prevent thrombus formation (e.g., within the left atrial appendage 10 and/or on each of the first piece of foam 120, the second piece of foam 130, the third piece of foam 140, etc.). In some embodiments, each of the first piece of foam 120, the second piece of foam 130, the third piece of foam 140, etc. may include an anti-thrombus agent(s) and/or medicament(s). In some embodiments, each of the first piece of foam 120, the second piece of foam 130, the third piece of foam 140, etc. may be configured to absorb blood and/or bodily fluid(s). In some embodiments, each of the first piece of foam 120, the second piece of foam 130, the third piece of foam 140, etc. may be configured to trap thrombus. In some embodiments, each of the first piece of foam 120, the second piece of foam 130, the third piece of foam 140, etc. may be configured to promote tissue ingrowth and/or endothelization. Other configurations are also contemplated.

In some embodiments, each of the first piece of foam 120, the second piece of foam 130, the third piece of foam 140, etc. may be unconstrained by outside forces and/or structure(s) in the first configuration (e.g., the collapsed configuration). In some embodiments, each of the first piece of foam 120, the second piece of foam 130, the third piece of foam 140, etc. may be self-maintained in the first configuration (e.g., the collapsed configuration) by shape memory properties. Other configurations are also contemplated.

In some embodiments, each of the first piece of foam 120, the second piece of foam 130, the third piece of foam 140, etc. may optionally comprise a radiopaque marker coupled thereto and/or embedded therein. In some embodiments, the radiopaque marker may comprise a radiopaque substance or a radiopaque material disposed within the first piece of foam 120, the second piece of foam 130, the third piece of foam 140, etc. (e.g., the first piece of foam 120, the second piece of foam 130, the third piece of foam 140, etc. may be doped with and/or may include the radiopaque substance or the radiopaque material).

Additional pieces of foam, locking structures, etc. may also be used as desired for occluding the left atrial appendage 10. The device 100 and system 200 disclosed herein may be considered to be modular, in that additional elements and/or different sizes of elements may be added and/or used as needed to achieve desired results.

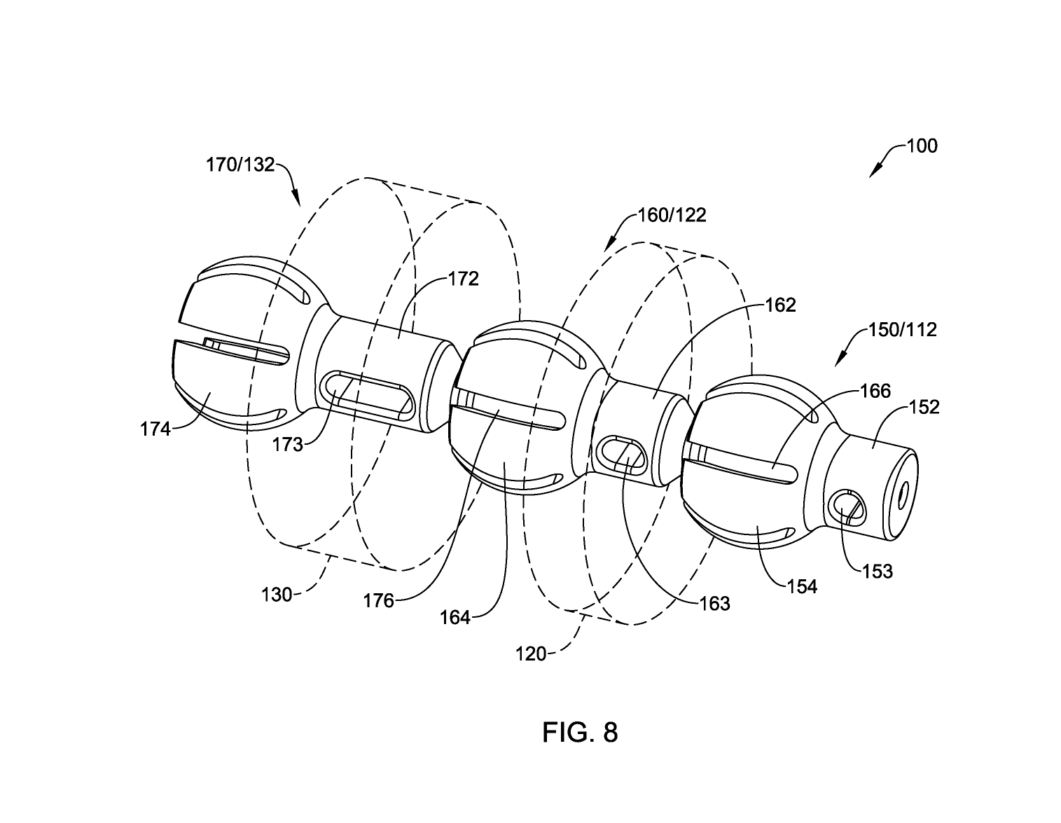

FIGS. 8-23 illustrate selected aspects of various embodiments of locking structures associated with the disclosure. It shall be understood that the various embodiments of locking structures may be used in the device 100 and the system 200 illustrated in FIGS. 1 and 5-7. Similarly, it shall be understood that the various embodiments of locking structures may also be used in various combinations with the implant anchor element 110 and/or embodiments thereof shown in FIGS. 2A-4. Some elements and/or reference numbers are discussed herein for context and may be better understood and/or further described with respect to other figures, as all reference numbers discussed do not necessarily appear in the figures being described.

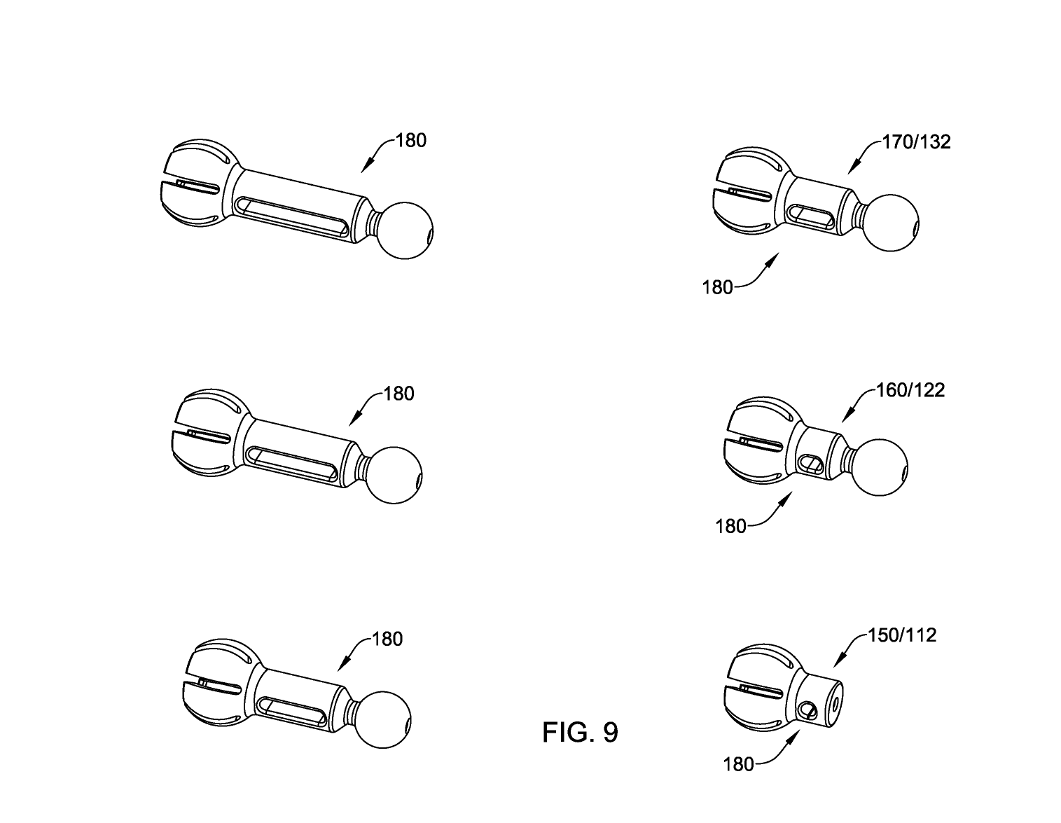

FIGS. 8-9 illustrate selected aspects of an embodiment of the device 100. For the purpose of illustration only, the device 100 in FIG. 8 is shown with a first locking structure 150 that may correspond to and/or may be the first locking structure 112 (without the implant anchor element 110 fixedly attached thereto), a second locking structure 160 that may correspond to and/or may be the second locking structure 122 engaged with the first locking structure 150, and a third locking structure 170 that may correspond to and/or may be the third locking structure 132 engaged with the second locking structure 160. Other configurations and/or elements are also contemplated.

In some embodiments, the first locking structure 150 may comprise a body portion 152 and a receiver portion 154 disposed at a proximal end of the body portion 152. In at least some embodiments, the body portion 152 may be monolithically formed with the receiver portion 154. The body portion 152 may be fixedly attached to the implant anchor element 110 (e.g., FIG. 1) at a distal end of the body portion 152. In some alternative embodiments, the body portion 152 may comprise one or more cavities 153 configured to receive a portion of a piece of foam (not shown) therein. The receiver portion 154 may be configured to engage with the second locking structure 160. In some embodiments, the receiver portion 154 may comprise a plurality of longitudinally oriented tabs forming a cup-shaped receptacle. In some embodiments, the plurality of longitudinally oriented tabs may be self-biased toward a locked position. In some embodiments, the plurality of longitudinally oriented tabs may be resiliently deflectable radially outward to permit the second locking structure 160 to be received within the cup-shaped receptacle.

In some embodiments, the second locking structure 160 may comprise a body portion 162, a receiver portion 164 disposed at a proximal end of the body portion 162, and an insertion portion 166 disposed at a distal end of the body portion 162. In at least some embodiments, the body portion 162 may be monolithically formed with the receiver portion 164 and the insertion portion 166. In some embodiments, the body portion 162 may comprise one or more cavities 163 configured to receive a portion of the first piece of foam 120 therein. The receiver portion 164 may be configured to engage with the third locking structure 170. In some embodiments, the receiver portion 164 may comprise a plurality of longitudinally oriented tabs forming a cup-shaped receptacle. In some embodiments, the plurality of longitudinally oriented tabs may be self-biased toward a locked position. In some embodiments, the plurality of longitudinally oriented tabs may be resiliently deflectable radially outward to permit the third locking structure 170 to be received within the cup-shaped receptacle. In some alternative configurations, the second locking structure 160 may be devoid of the receiver portion 164, such that the second locking structure 160 forms a cap of the device 100 with a closed proximal end that is configured to cooperate with the first piece of foam 120 to seal off the left atrial appendage 10 (e.g., FIG. 5) from the left atrium of the patient’s heart.

In some embodiments, a first axial force applied to the second delivery device 220 (e.g., FIG. 5) and/or the elongate shaft of the second delivery device 220 is required to translate the first piece of foam 120 through the lumen 204 of the guide sheath 202, and a second axial force applied to the second delivery device 220 and/or the elongate shaft of the second delivery device 220 is required to engage the second locking structure 122/160 with the first locking structure 112/150, wherein the second axial force applied to the second delivery device 220 and/or the elongate shaft of the second delivery device 220 is greater than the first axial force applied to the second delivery device 220 and/or the elongate shaft of the second delivery device 220.

In some embodiments, the third locking structure 170 may comprise a body portion 172, a receiver portion 174 disposed at a proximal end of the body portion 172, and an insertion portion 176 disposed at a distal end of the body portion 172. In at least some embodiments, the body portion 172 may be monolithically formed with the receiver portion 174 and the insertion portion 176. In some embodiments, the body portion 172 may comprise one or more cavities 173 configured to receive a portion of the second piece of foam 130 therein. The receiver portion 174 may be configured to engage with the fourth locking structure 142 (e.g., FIG. 7). In some embodiments, the receiver portion 174 may comprise a plurality of longitudinally oriented tabs forming a cup-shaped receptacle. In some embodiments, the plurality of longitudinally oriented tabs may be self-biased toward a locked position. In some embodiments, the plurality of longitudinally oriented tabs may be resiliently deflectable radially outward to permit the fourth locking structure 142 to be received within the cup-shaped receptacle. In some alternative configurations, the third locking structure 170 may be devoid of the receiver portion 174, such that the third locking structure 170 forms a cap of the device 100 with a closed proximal end that is configured to cooperate with the second piece of foam 130 to seal off the left atrial appendage 10 (e.g., FIG. 6) from the left atrium of the patient’s heart.

In some embodiments, a first axial force applied to the third delivery device 230 (e.g., FIG. 6) and/or the elongate shaft of the third delivery device 230 is required to translate the second piece of foam 130 through the lumen 204 of the guide sheath 202, and a second axial force applied to the third delivery device 230 and/or the elongate shaft of the third delivery device 230 is required to engage the third locking structure 132/170 with the second locking structure 122/160, wherein the second axial force applied to the third delivery device 230 and/or the elongate shaft of the third delivery device 230 is greater than the first axial force applied to the third delivery device 230 and/or the elongate shaft of the third delivery device 230.

In some embodiments, a first axial force applied to the fourth delivery device 240 (e.g., FIG. 7) and/or the elongate shaft of the fourth delivery device 240 is required to translate the third piece of foam 140 through the lumen 204 of the guide sheath 202, and a second axial force applied to the fourth delivery device 240 and/or the elongate shaft of the fourth delivery device 240 is required to engage the fourth locking structure 142 (e.g., FIG. 7) with the third locking structure 132/170, wherein the second axial force applied to the fourth delivery device 240 and/or the elongate shaft of the fourth delivery device 240 is greater than the first axial force applied to the fourth delivery device 240 and/or the elongate shaft of the fourth delivery device 240.

FIG. 9 illustrates different example configurations for individual elements of the device 100 and/or the system 200. In some embodiments, the system 200 may comprise and/or may be a kit consisting of a plurality of locking structures 180 constructed in accordance with the disclosure, along with associated delivery devices, etc. As shown in FIG. 9, the locking structures of FIG. 8 and/or the plurality of locking structures 180 may be made and/or used in different sizes and/or configurations that may be mixed and/or matched as needed to close off the left atrial appendage of the patient. Additionally, in some embodiments, multiple instances of the same locking structure may be used. Labels and/or reference numbers affixed to the locking structures of FIG. 9 are merely exemplary and are not intended to be limiting. The locking structures of FIGS. 8-9 may be configured to and/or considered to passively lock together, such as via a snap fit for example.

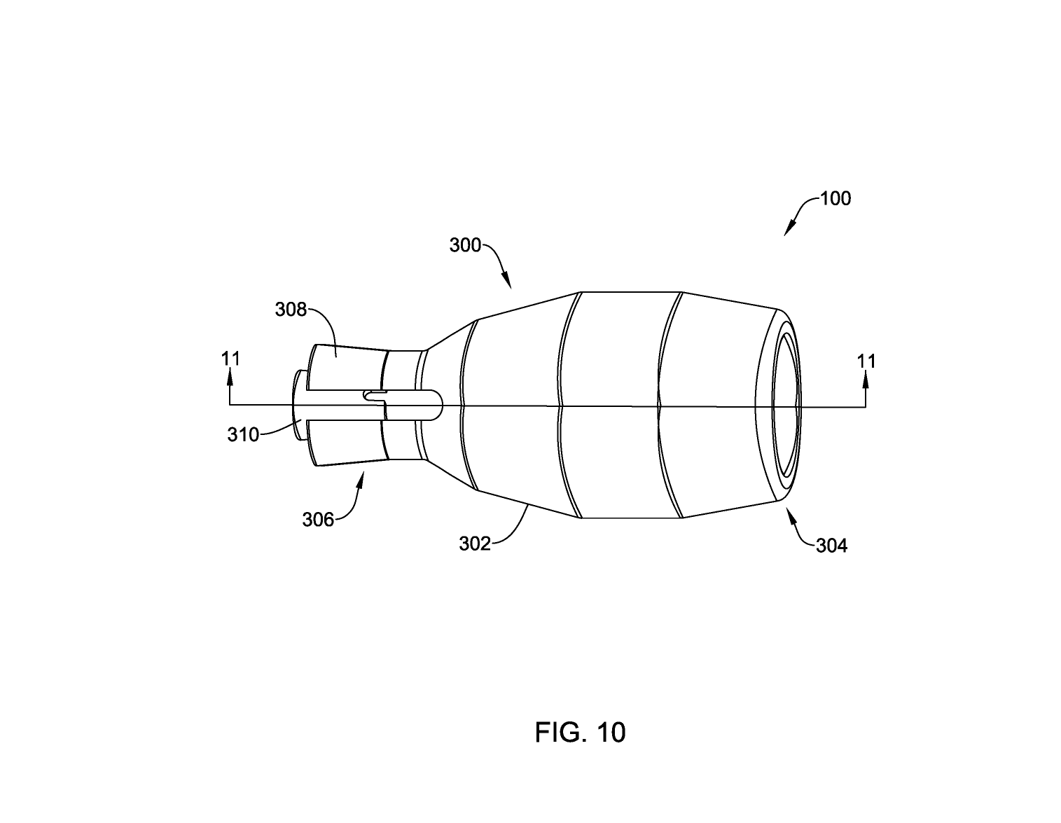

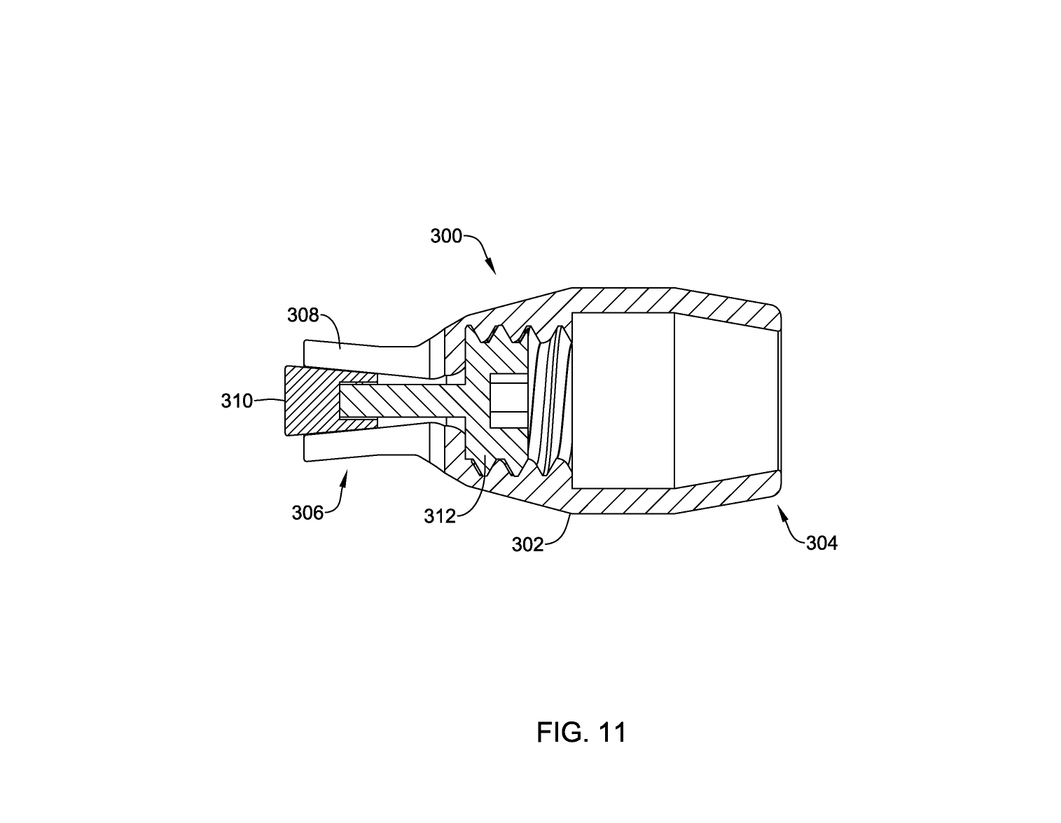

FIGS. 10-13 illustrate selected aspects of an embodiment of the device 100. FIGS. 10-11 illustrate an example locking structure 300 that may correspond to and/or may be used as the first locking structure 112, the second locking structure 122, the third locking structure 132, the fourth locking structure 142, etc.

In some embodiments, the locking structure 300 may comprise a body portion 302, a receiver portion 304 disposed at a proximal end of the body portion 302, and an insertion portion 306 disposed at a distal end of the body portion 302. In at least some embodiments, the body portion 302 may be monolithically formed with the receiver portion 304 and the insertion portion 306. The receiver portion 304 may be configured to engage with the insertion portion 306 of an adjacent (e.g., a second) locking structure 300. In some embodiments, the receiver portion 304 may comprise a cup-shaped receptacle. In some embodiments, the insertion portion 306 may comprise a plurality of longitudinally oriented tabs 308 that may be self-biased toward a collapsed position. In some embodiments, the plurality of longitudinally oriented tabs 308 may be resiliently deflectable radially outward toward and/or to an expanded position by a wedge member 310 (e.g., FIG. 11) disposed therein to actively lock the insertion portion 306 to the receiver portion 304 of the adjacent locking structure 300 when the insertion portion 306 is disposed within the cup-shaped receptacle. In some embodiments, the wedge member 310 may be operatively coupled to a threaded member 312 disposed within and/or engaged with the body portion 302. In some embodiments, a first portion (e.g., the wedge member 310) of the locking structure 300 may be movable axially relative to a second portion (e.g., the insertion portion 306 and/or the plurality of longitudinally oriented tabs 308) of the locking structure 300 to lock the locking structure 300 to an adjacent locking structure 300. For example, as the threaded member 312 is rotated relative to the body portion 302, a longitudinal position of the wedge member 310 relative to the insertion portion 306 and/or the plurality of longitudinally oriented tabs 308 may change, thereby causing the plurality of longitudinally oriented tabs 308 to move radially relative to the body portion 302.

In some embodiments, one or more locking structures 300 may comprise different features and/or may omit certain elements or features. For example, while not expressly illustrated, a first locking structure 300 (e.g., ref. 112) may be devoid of the insertion portion 306, the plurality of longitudinally oriented tabs 308, and the wedge member 310 such that the first locking structure 300 and/or the body portion 302 of the first locking structure 300 may be fixedly attached to the implant anchor element 110 as discussed herein.

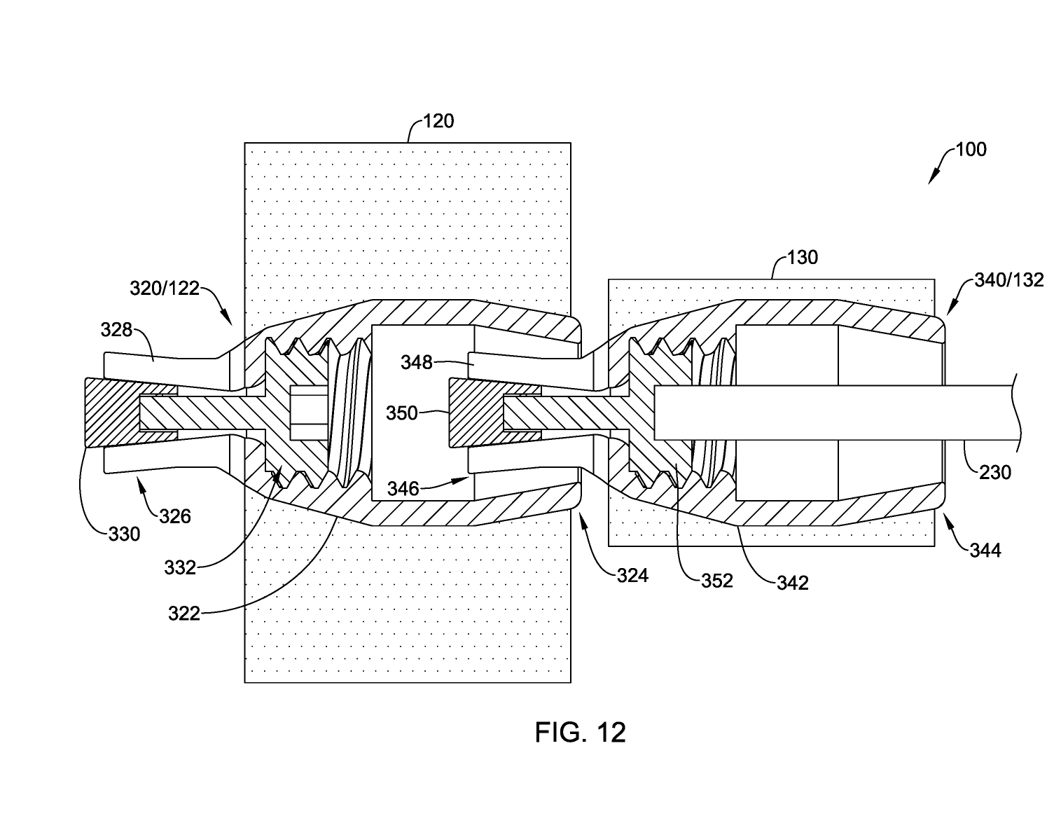

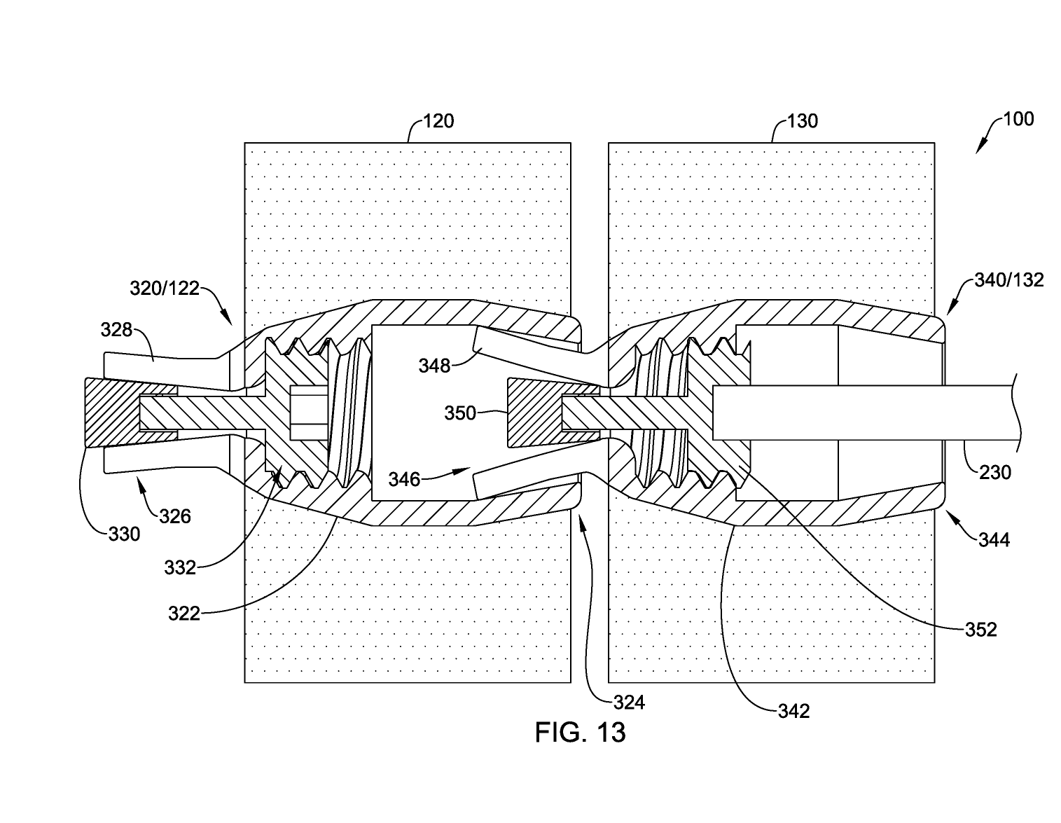

FIGS. 12-13 are cross-sectional views illustrating interaction and active locking between two adjacent locking structures. For the purpose of illustration and discussion, the adjacent locking structures shown in FIGS. 12-13 may include a second locking structure 320 (e.g., ref. 122) and a third locking structure 340 (e.g., ref. 132). Additional locking structures may be used, provided, and/or included in accordance with the disclosure. It shall be understood that in at least some embodiments, the following discussion may apply to all adjacent locking structures present in the device 100, including engaging the second locking structure 320 with the first locking structure 300.

In some embodiments, the second locking structure 320 may comprise a body portion 322, a receiver portion 324 disposed at a proximal end of the body portion 322, and an insertion portion 326 disposed at a distal end of the body portion 322. In at least some embodiments, the body portion 322 may be monolithically formed with the receiver portion 324 and the insertion portion 326. A first piece of foam 120 may be fixedly attached to the second locking structure 320 (e.g., ref. 122) and/or the body portion 322. While not shown in FIGS. 12-13, the insertion portion 326 may be insertable into the receiver portion 304 of the first locking structure 300 (e.g., ref. 112). The receiver portion 324 may be configured to engage with the third locking structure 340 (e.g., ref. 132). In some embodiments, the receiver portion 324 may comprise a cup-shaped receptacle. In some embodiments, the insertion portion 326 may comprise a plurality of longitudinally oriented tabs 328 that may be self-biased toward a collapsed position. In some embodiments, the plurality of longitudinally oriented tabs 328 may be resiliently deflectable radially outward toward and/or to an expanded position by a wedge member 330 disposed therein to actively lock the insertion portion 326 to the receiver portion 304 of the first locking structure 300 when the insertion portion 326 is disposed within the receiver portion 304 and/or the cup-shaped receptacle of the first locking structure 300. In some embodiments, the wedge member 330 may be operatively coupled to a threaded member 332 disposed within and/or engaged with the body portion 322. In some embodiments, a first portion (e.g., the wedge member 330) of the second locking structure 320 may be movable axially relative to a second portion (e.g., the insertion portion 326 and/or the plurality of longitudinally oriented tabs 328) of the second locking structure 320 to lock the second locking structure 320 to the first locking structure 300. For example, as the threaded member 332 is rotated relative to the body portion 322, a longitudinal position of the wedge member 330 relative to the insertion portion 326 and/or the plurality of longitudinally oriented tabs 328 may change, thereby causing the plurality of longitudinally oriented tabs 328 to move radially relative to the body portion 322. In some alternative configurations, the second locking structure 320 may be devoid of the receiver portion 324, such that the second locking structure 320 forms a cap of the device 100 with a closed proximal end that is configured to cooperate with the first piece of foam 120 to seal off the left atrial appendage 10 (e.g., FIG. 5) from the left atrium of the patient’s heart.

Similar to above, the second delivery device (not shown; ref. 220) and/or the elongate shaft of the second delivery device may be releasably coupled to the first piece of foam 120 and/or the second locking structure 320 (e.g., ref. 122). In some embodiments, a first axial force applied to the second delivery device and/or the elongate shaft of the second delivery device is required to translate the first piece of foam 120 through the lumen 204 of the guide sheath 202, and a second axial force applied to the second delivery device and/or the elongate shaft of the second delivery device is required to engage the second locking structure 122/320 with the first locking structure 112/300. In at least some embodiments, the second axial force applied to the second delivery device and/or the elongate shaft of the second delivery device may be similar to and/or substantially the same as the first axial force applied to the second delivery device and/or the elongate shaft of the second delivery device when the plurality of longitudinally oriented tabs 328 is disposed in the collapsed position. In the collapsed position, the plurality of longitudinally oriented tabs 328 may be axially movable into and/or out of the receiver portion 304 of the first locking structure 300 with very little additional effort beyond that required to translate the first piece of foam 120. After positioning the insertion portion 326 of the second locking structure 320 (e.g., ref. 122) into the receiver portion 304 of the first locking structure 300 (e.g., ref. 112), the second delivery device and/or the elongate shaft of the second delivery device may be rotated relative to the second locking structure 320 and/or the body portion 322 to rotate the threaded member 332 relative to the body portion 322 and shift a longitudinal position of the wedge member 330 relative to the insertion portion 326 and/or the plurality of longitudinally oriented tabs 328, thereby causing the plurality of longitudinally oriented tabs 328 to move radially relative to the body portion 322 (e.g., to shift from the collapsed position radially outward toward and/or to the expanded position) to actively lock the second locking structure 320 (e.g., ref. 122) to the first locking structure 300 (e.g., ref. 112). After locking the second locking structure 320 to the first locking structure 300, the second delivery device and/or an elongate shaft of the second delivery device may be decoupled from the second locking structure 320 and removed.

Turning now more specifically to FIGS. 12-13, in some embodiments, the third locking structure 340 may comprise a body portion 342, a receiver portion 344 disposed at a proximal end of the body portion 342, and an insertion portion 346 disposed at a distal end of the body portion 342. In at least some embodiments, the body portion 342 may be monolithically formed with the receiver portion 344 and the insertion portion 346. A second piece of foam 130 may be fixedly attached to the second locking structure 340 (e.g., ref. 132) and/or the body portion 342. The insertion portion 346 may be insertable into the receiver portion 324 of the second locking structure 320 (e.g., ref. 122), as seen in FIG. 12. The receiver portion 344 may be configured to engage with a fourth locking structure (e.g., ref. 142). In some embodiments, the receiver portion 344 may comprise a cup-shaped receptacle. In some embodiments, the insertion portion 346 may comprise a plurality of longitudinally oriented tabs 348 that may be self-biased toward a collapsed position. In some embodiments, the plurality of longitudinally oriented tabs 348 may be resiliently deflectable radially outward toward and/or to an expanded position by a wedge member 350 disposed therein to actively lock the insertion portion 346 to the receiver portion 324 of the second locking structure 320 when the insertion portion 346 is disposed within the receiver portion 324 and/or the cup-shaped receptacle of the second locking structure 320. In some embodiments, the wedge member 350 may be operatively coupled to a threaded member 352 disposed within and/or engaged with the body portion 342. In some embodiments, a first portion (e.g., the wedge member 350) of the third locking structure 340 may be movable axially relative to a second portion (e.g., the insertion portion 346 and/or the plurality of longitudinally oriented tabs 348) of the third locking structure 340 to lock the third locking structure 340 to the second locking structure 320. As the threaded member 352 is rotated relative to the body portion 342, a longitudinal position of the wedge member 350 relative to the insertion portion 346 and/or the plurality of longitudinally oriented tabs 348 may change, thereby causing the plurality of longitudinally oriented tabs 348 to move radially relative to the body portion 342. In some alternative configurations, the third locking structure 340 may be devoid of the receiver portion 344, such that the third locking structure 340 forms a cap of the device 100 with a closed proximal end that is configured to cooperate with the second piece of foam 130 to seal off the left atrial appendage 10 (e.g., FIG. 6) from the left atrium of the patient’s heart.

Similar to above, the third delivery device 230 and/or the elongate shaft of the third delivery device 230 may be releasably coupled to the second piece of foam 130 and/or the third locking structure 340 (e.g., ref. 132). In some embodiments, a first axial force applied to the third delivery device 230 and/or the elongate shaft of the third delivery device 230 is required to translate the second piece of foam 130 through the lumen 204 of the guide sheath 202, and a second axial force applied to the third delivery device 230 and/or the elongate shaft of the third delivery device 230 is required to engage the third locking structure 132/340 with the second locking structure 122/320. In at least some embodiments, the second axial force applied to the third delivery device 230 and/or the elongate shaft of the third delivery device 230 may be similar to and/or substantially the same as the first axial force applied to the third delivery device 230 and/or the elongate shaft of the third delivery device 230 when the plurality of longitudinally oriented tabs 348 is disposed in the collapsed position. In the collapsed position, the plurality of longitudinally oriented tabs 348 may be axially movable into and/or out of the receiver portion 324 of the second locking structure 320 with very little additional effort beyond that required to translate the second piece of foam 130. After positioning the insertion portion 346 of the third locking structure 340 (e.g., ref. 132) into the receiver portion 324 of the second locking structure 320 (e.g., ref. 122), the third delivery device 230 and/or the elongate shaft of the third delivery device 230 may be rotated relative to the third locking structure 340 and/or the body portion 342 to rotate the threaded member 352 relative to the body portion 342 and shift a longitudinal position of the wedge member 350 relative to the insertion portion 346 and/or the plurality of longitudinally oriented tabs 348, thereby causing the plurality of longitudinally oriented tabs 348 to move radially relative to the body portion 342 (e.g., to shift from the collapsed position radially outward toward and/or to the expanded position) to actively lock the third locking structure 340 (e.g., ref. 132) to the second locking structure 320 (e.g., ref. 122), as seen in FIG. 13.

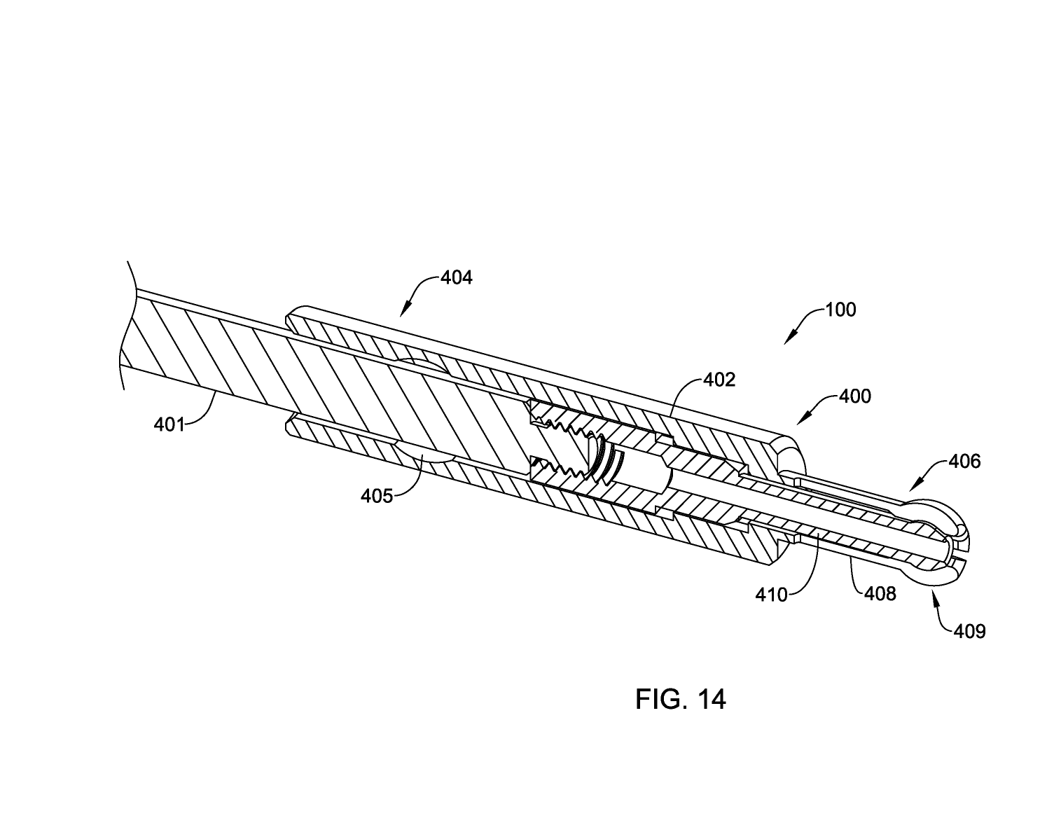

FIGS. 14-18 illustrate selected aspects of an embodiment of the device 100. FIG. 14 illustrates selected aspects of a locking structure 400 that may correspond to and/or may be used as the first locking structure 112, the second locking structure 122, the third locking structure 132, the fourth locking structure 142, etc.

In some embodiments, the locking structure 400 may comprise a body portion 402, a receiver portion 404 disposed at a proximal end of the body portion 402, and an insertion portion 406 disposed at a distal end of the body portion 402. In at least some embodiments, the body portion 402 may be monolithically formed with the receiver portion 404 and the insertion portion 406. The receiver portion 404 may be configured to engage with the insertion portion 406 of an adjacent (e.g., a second) locking structure 400. In some embodiments, the receiver portion 404 may comprise a receptacle and a bulbous recess 405 extending radially outward from and/or within an inner wall of the receptacle. In some embodiments, the insertion portion 406 may comprise a plurality of longitudinally oriented tabs 408 that may be self-biased toward a collapsed position. The plurality of longitudinally oriented tabs 408 may comprise and/or may form a rounded tip 409 extending radially outward therefrom. In some embodiments, the plurality of longitudinally oriented tabs 408 and/or the rounded tip 409 may be resiliently deflectable radially outward toward and/or to an expanded position by a locking pin 410 disposed therein to actively lock the insertion portion 406 to the receiver portion 404 of the adjacent locking structure 400 when the insertion portion 406 is disposed within the receptacle and/or the rounded tip 409 is disposed within the bulbous recess 405. In some embodiments, the locking pin 410 may be operatively coupled to a delivery device 401 and/or an elongate shaft of a delivery device 401. The delivery device 401 may be considered to correspond to and/or may be any delivery device disclosed herein – including but not limited to the delivery device(s) 210/220/230/240. In some embodiments, the locking pin 410 may be axially movable within the insertion portion 406 via axial translation and/or axial actuation of the delivery device 401 coupled thereto and/or associated therewith. In some embodiments, a first portion (e.g., the locking pin 410) of the locking structure 400 may be movable axially relative to a second portion (e.g., the insertion portion 406 and/or the plurality of longitudinally oriented tabs 408) of the locking structure 400 to lock the locking structure 400 to an adjacent locking structure 400.

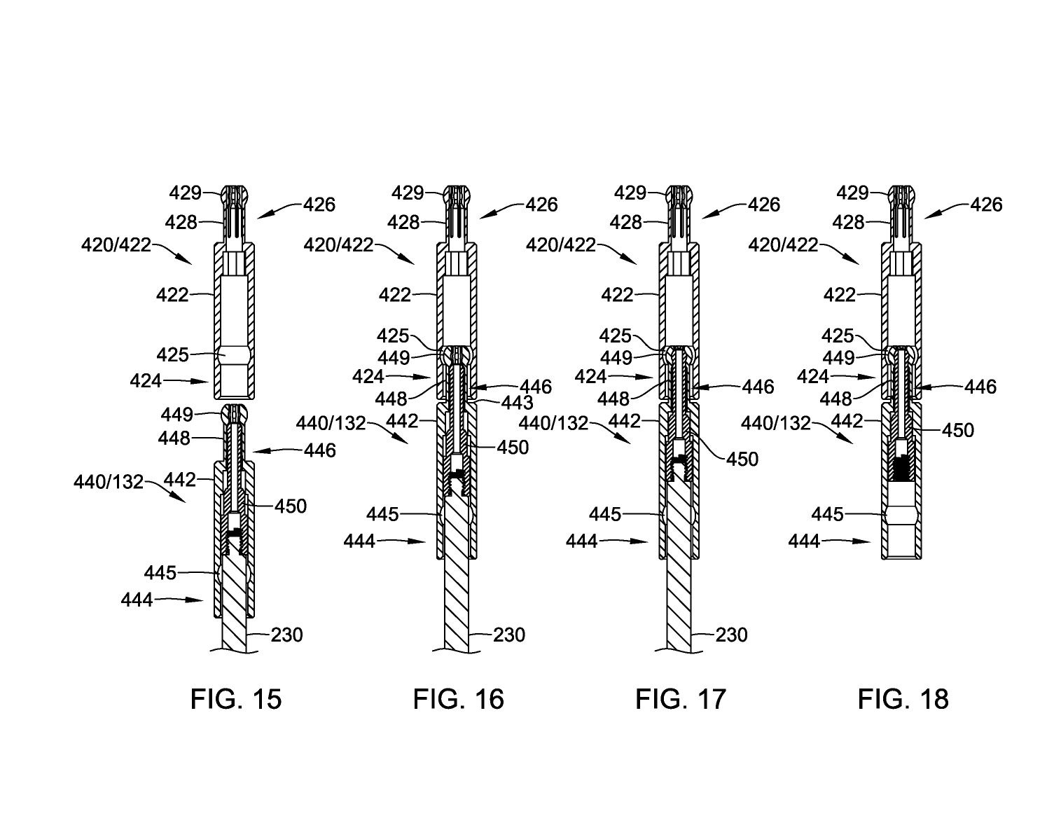

FIGS. 15-18 are cross-sectional views illustrating interaction and active locking between two adjacent locking structures 400. In some embodiments, one or more locking structures 400 may comprise different features and/or may omit certain elements or features. For example, while not expressly illustrated, a first locking structure 400 (e.g., ref. 112) may be devoid of the insertion portion 406, the plurality of longitudinally oriented tabs 408, and the locking pin 410 such that the first locking structure 400 and/or the body portion 402 of the first locking structure 400 may be fixedly attached to the implant anchor element 110 as discussed herein.

For the purpose of illustration and discussion, the adjacent locking structures 400 shown in FIGS. 15-18 may include a second locking structure 420 (e.g., ref. 122) and a third locking structure 440 (e.g., ref. 132). Additional locking structures may be used, provided, and/or included in accordance with the disclosure. It shall be understood that in at least some embodiments, the following discussion may apply to all adjacent locking structures 400 present in the device 100, including engaging the second locking structure 420 with the first locking structure 400. Some elements or features are not shown to improve clarity.

In some embodiments, the second locking structure 420 may comprise a body portion 422, a receiver portion 424 disposed at a proximal end of the body portion 422, and an insertion portion 426 disposed at a distal end of the body portion 422. In at least some embodiments, the body portion 422 may be monolithically formed with the receiver portion 424 and the insertion portion 426. A first piece of foam 120 (not shown) may be fixedly attached to the second locking structure 420 (e.g., ref. 122) and/or the body portion 422. The insertion portion 426 may be insertable into the receiver portion 404 of the first locking structure 400 (e.g., ref. 112). The receiver portion 424 may be configured to engage with the third locking structure 440 (e.g., ref. 132). In some embodiments, the receiver portion 424 may comprise a receptacle and a bulbous recess 425 extending radially outward from and/or within an inner wall of the receptacle. In some embodiments, the insertion portion 426 may comprise a plurality of longitudinally oriented tabs 428 that may be self-biased toward a collapsed position. The plurality of longitudinally oriented tabs 428 may comprise and/or may form a rounded tip 429 extending radially outward therefrom. In some embodiments, the plurality of longitudinally oriented tabs 428 and/or the rounded tip 429 may be resiliently deflectable radially outward toward and/or to an expanded position by a locking pin (not shown) disposed therein to actively lock the insertion portion 426 to the receiver portion 404 of the first locking structure 400 when the insertion portion 426 is disposed within the receptacle and/or the rounded tip 429 is disposed within the bulbous recess 405 (e.g., FIG. 14). In some embodiments, the locking pin may be operatively coupled to the second delivery device (not shown) and/or the elongate shaft of the second delivery device. In some embodiments, a first portion (e.g., the locking pin (not shown)) of the second locking structure 420 may be movable axially relative to a second portion (e.g., the insertion portion 426 and/or the plurality of longitudinally oriented tabs 428) of the second locking structure 420 to lock the second locking structure 420 to the first locking structure 400. For example, in some embodiments, the locking pin may be axially movable within the insertion portion 426 in a distal direction via axial translation and/or axial actuation of the second delivery device and/or the elongate shaft of the second delivery device coupled thereto and/or associated therewith to actively lock the second locking structure 420 (e.g., ref. 122) to the first locking structure 400 (e.g., ref. 112). After locking the second locking structure 420 to the first locking structure 400, the second delivery device and/or the elongate shaft of the second delivery device may be decoupled from the second locking structure 420 and removed.

Turning now more specifically to FIGS. 15-18, in some embodiments, the third locking structure 440 may comprise a body portion 442, a receiver portion 444 disposed at a proximal end of the body portion 442, and an insertion portion 446 disposed at a distal end of the body portion 442. In at least some embodiments, the body portion 442 may be monolithically formed with the receiver portion 444 and the insertion portion 446. A second piece of foam 130 (not shown) may be fixedly attached to the third locking structure 440 (e.g., ref. 132) and/or the body portion 442. The insertion portion 446 may be insertable into the receiver portion 424 of the second locking structure 420 (e.g., ref. 112). The receiver portion 444 may be configured to engage with a fourth locking structure (e.g., ref. 142). In some embodiments, the receiver portion 444 may comprise a receptacle and a bulbous recess 445 extending radially outward from and/or within an inner wall of the receptacle. In some embodiments, the insertion portion 446 may comprise a plurality of longitudinally oriented tabs 448. In some embodiments, the plurality of longitudinally oriented tabs 448 may be self-biased toward a collapsed position. The plurality of longitudinally oriented tabs 448 may comprise and/or may form a rounded tip 449 extending radially outward therefrom. In some embodiments, the rounded tip 449 may engage with the inner wall of the receptacle of the receiver portion 424 of the second locking structure 420 as the third locking structure 440 is advanced and/or translated distally to radially collapse the insertion portion 446 and/or the plurality of longitudinally oriented tabs 448 toward and/or to the collapsed position, thereby permitting the insertion portion 446 and/or the rounded tip 449 to be advanced into the receiver portion 424 and/or the receptacle of the second locking structure 420. In some embodiments, the rounded tip 449 may have a radial extent in the collapsed position that is smaller than a radial extent of the receptacle, thereby permitting the insertion portion 446 and/or the rounded tip 449 to be advanced into the receiver portion 424 and/or the receptacle of the second locking structure 420. The insertion portion 446 and/or the rounded tip 449 may be advanced into the receiver portion 424 and/or the receptacle of the second locking structure 420 until a distal shoulder 443 of the body portion 442 engages and/or contacts a proximal shoulder and/or a proximal end of the receiver portion 424 of the second locking structure 420, as seen in FIG. 16.

In some embodiments, the plurality of longitudinally oriented tabs 448 and/or the rounded tip 449 may be resiliently deflectable radially outward toward and/or to an expanded position within the bulbous recess 445 by a locking pin 450 disposed therein to actively lock the insertion portion 446 to the receiver portion 424 of the second locking structure 420 when the insertion portion 446 is disposed within the receptacle of the second locking structure 420 and/or the rounded tip 449 is disposed within the bulbous recess 425 of the second locking structure 420. In some embodiments, the locking pin 450 may be operatively coupled to the third delivery device 230 and/or the elongate shaft of the third delivery device 230. In some embodiments, a first portion (e.g., the locking pin 450) of the third locking structure 440 may be movable axially relative to a second portion (e.g., the insertion portion 446 and/or the plurality of longitudinally oriented tabs 448) of the third locking structure 440 to lock the third locking structure 440 to the second locking structure 420. For example, in some embodiments, the locking pin 450 may be axially movable within and/or relative to the insertion portion 446 in a distal direction via axial translation and/or axial actuation of the third delivery device 230 and/or the elongate shaft of the third delivery device 230 coupled thereto and/or associated therewith to actively lock the third locking structure 440 (e.g., ref. 132) to the second locking structure 420 (e.g., ref. 122), as seen in FIG. 17. In some embodiments, the radial extent of the rounded tip 449 in the expanded position may be greater than the radial extent of the receptacle, thereby preventing the insertion portion 446 and/or the rounded tip 449 from being removed from the receiver portion 424 and/or the receptacle of the second locking structure 420. In some embodiments, the locking pin 450 may prevent radial compression of the insertion portion 446 and/or the plurality of longitudinally oriented tabs 448 such that the rounded tip 449 cannot be removed from the receiver portion 424 and/or the receptacle of the second locking structure 420 without damaging and/or breaking the second locking structure 420 and/or the third locking structure 440. Thereafter, the third delivery device and/or the elongate shaft of the third delivery device 230 may be decoupled from the third locking structure 440 and removed, as seen in FIG. 18.

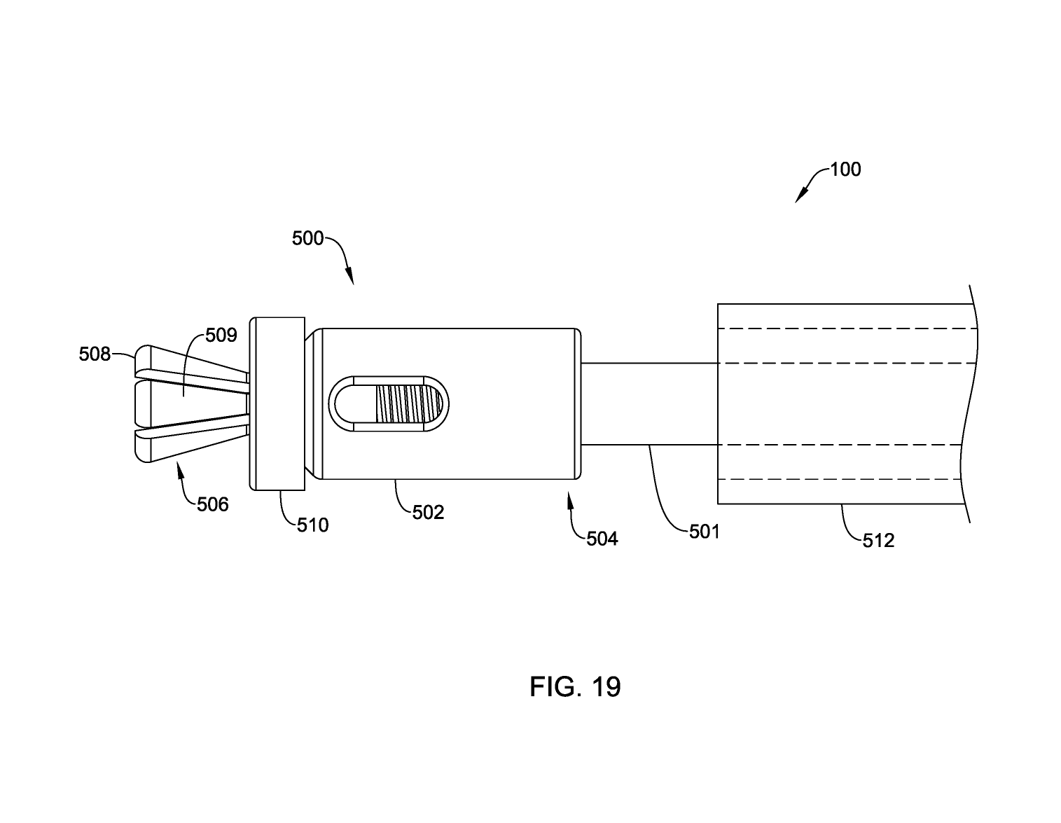

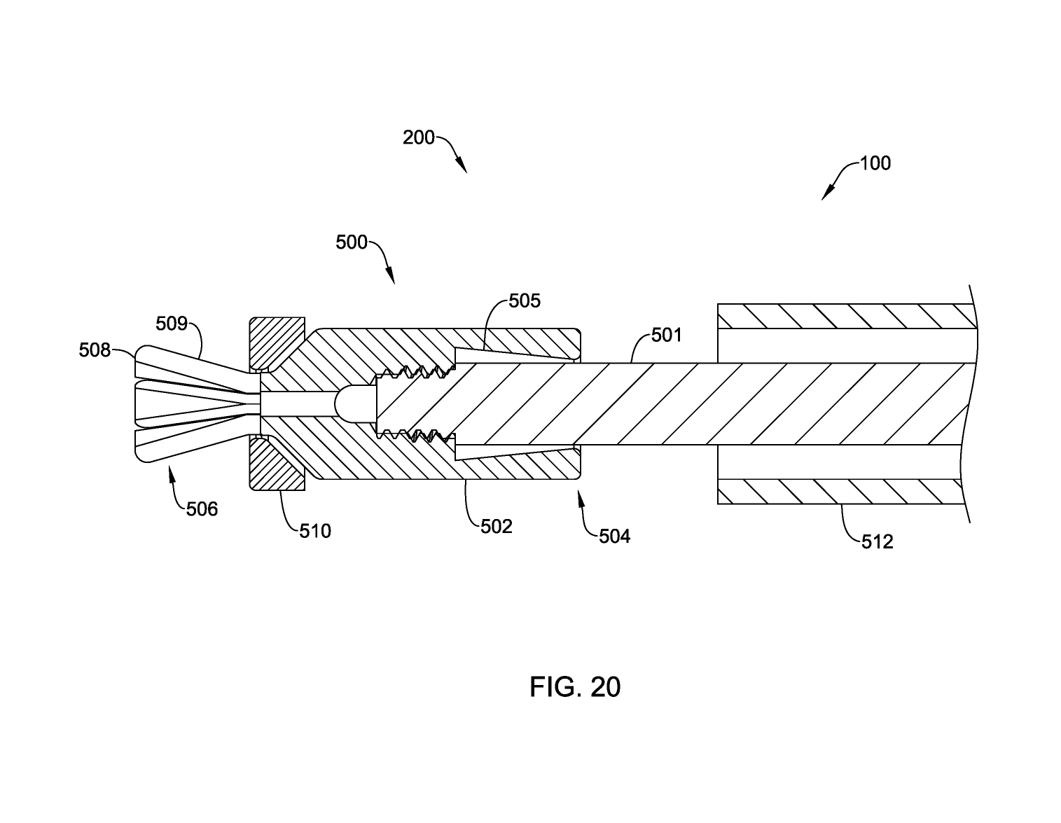

FIGS. 19-20 illustrate selected aspects of an embodiment of the device 100. FIG. 19 illustrates selected aspects of a locking structure 500 that may correspond to and/or may be used as the first locking structure 112, the second locking structure 122, the third locking structure 132, the fourth locking structure 142, etc. FIG. 20 is a cross-sectional view of the locking structure 500 of FIG. 19.

In some embodiments, the locking structure 500 may comprise a body portion 502, a receiver portion 504 disposed at a proximal end of the body portion 502, and an insertion portion 506 disposed at a distal end of the body portion 502. In at least some embodiments, the body portion 502 may be monolithically formed with the receiver portion 504 and the insertion portion 506. The receiver portion 504 of the first locking structure 500 may be configured to engage with the insertion portion 506 of an adjacent (e.g., a second) locking structure 500. In some embodiments, the receiver portion 504 may comprise a receptacle having an inner surface 505 (e.g., FIG. 20) that is angled radially outward in a distal direction (e.g., toward the insertion portion 506). In some embodiments, the insertion portion 506 may comprise a plurality of longitudinally oriented tabs 508 that may be self-biased toward an expanded position. In some embodiments, each longitudinally oriented tab of the plurality of longitudinally oriented tabs 508 may comprise an outer surface 509 that is angled radially outward in the distal direction. In some embodiments, the plurality of longitudinally oriented tabs 508 may comprise a collective outer surface that is circumferentially discontinuous and is angled radially outward in the distal direction. In one example, the collective outer surface may be substantially conical in shape.

In some embodiments, the plurality of longitudinally oriented tabs 508 may be resiliently deflectable radially inward toward and/or to a collapsed position by a ring member 510 movably disposed around the plurality of longitudinally oriented tabs 508 to permit the insertion portion 506 and/or the plurality of longitudinally oriented tabs 508 to be inserted into the receiver portion 504 of the adjacent locking structure 500. In some embodiments, the ring member 510 may be actuatable in a distal direction relative to the body portion 502, the insertion portion 506, and/or the plurality of longitudinally oriented tabs 508 by a tubular member 512 slidably disposable over the body portion 502 to shift the plurality of longitudinally oriented tabs 508 toward and/or to the collapsed position. With the plurality of longitudinally oriented tabs 508 in the collapsed position, the insertion portion 506 and/or the plurality of longitudinally oriented tabs 508 may be configured to be inserted into the receiver portion 504 of the adjacent locking structure 500. Thereafter, the tubular member 512 may be withdrawn, thereby permitting the ring member 510 to slide proximally along the insertion portion 506 and/or the plurality of longitudinally oriented tabs 508 such that the plurality of longitudinally oriented tabs 508 may radially expand toward and/or to the expanded position within the receptacle to engage the collective outer surface against the inner surface 505 of the receptacle to actively lock the insertion portion 506 to the receiver portion 504 of the adjacent locking structure 500 when the insertion portion 506 is disposed within the receptacle. In some embodiments, a first portion (e.g., the ring member 510) of the locking structure 500 may be movable axially relative to a second portion (e.g., the insertion portion 506 and/or the plurality of longitudinally oriented tabs 508) of the locking structure 500 to lock the locking structure 500 to an adjacent locking structure 500.

In some embodiments, the body portion 502 may be operatively coupled to a delivery device 501 and/or an elongate shaft of a delivery device 501. The delivery device 501 may be considered to correspond to and/or may be any delivery device disclosed herein – including but not limited to the delivery device(s) 210/220/230/240. The tubular member 512 may be slidably disposed over and/or around the delivery device 501 and/or the elongate shaft of the delivery device 501 in order to translate and/or actuate the ring member 510 relative to the body portion 502, the insertion portion 506, and/or the plurality of longitudinally oriented tabs 508. Other configurations are also contemplated.

In some embodiments, one or more locking structures 500 may comprise different features and/or may omit certain elements or features. For example, while not expressly illustrated, a first locking structure 500 (e.g., ref. 112) may be devoid of the insertion portion 506, the plurality of longitudinally oriented tabs 508, and the ring member 510 such that the first locking structure 500 and/or the body portion 502 of the first locking structure 500 may be fixedly attached to the implant anchor element 110 as discussed herein.

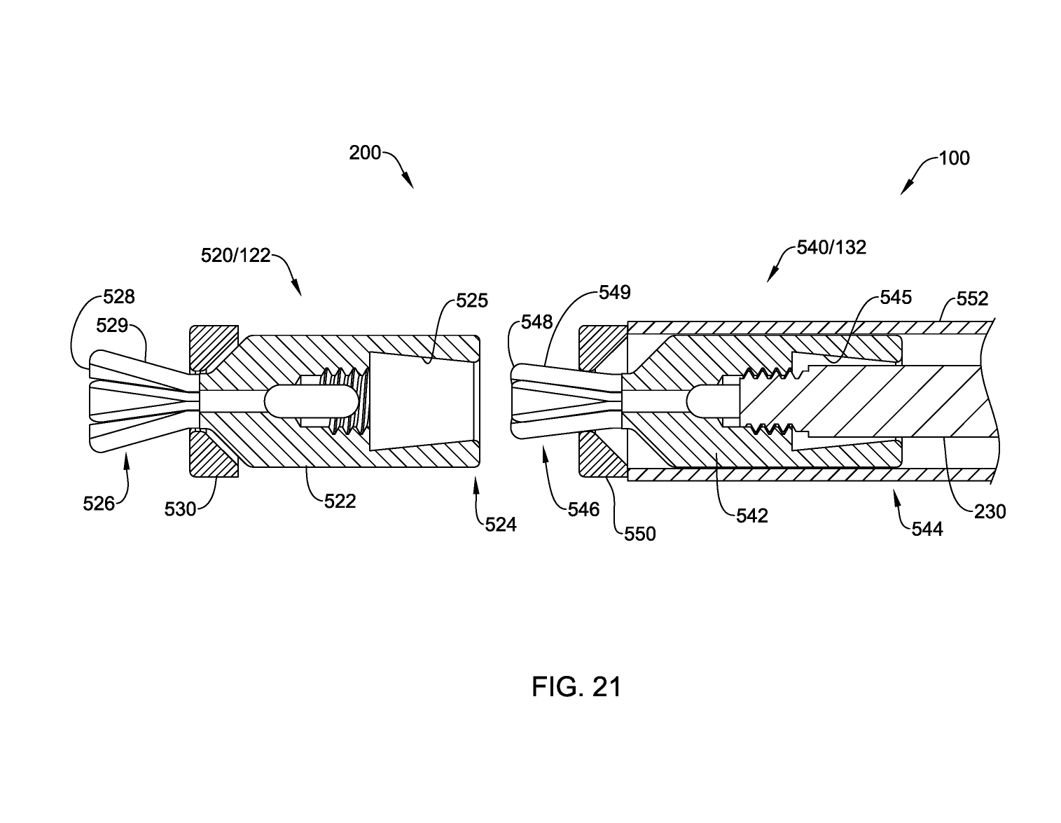

FIGS. 21-24 are cross-sectional views illustrating interaction and active locking between two adjacent locking structures. For the purpose of illustration and discussion, the adjacent locking structures shown in FIGS. 21-24 may include a second locking structure 520 (e.g., ref. 122) and a third locking structure 540 (e.g., ref. 132). Additional locking structures may be used, provided, and/or included in accordance with the disclosure. It shall be understood that in at least some embodiments, the following discussion may apply to all adjacent locking structures present in the device 100, including engaging the second locking structure 520 with the first locking structure 500. Some elements or features are not shown to improve clarity.

In some embodiments, the second locking structure 520 may comprise a body portion 522, a receiver portion 524 disposed at a proximal end of the body portion 522, and an insertion portion 526 disposed at a distal end of the body portion 522. In at least some embodiments, the body portion 522 may be monolithically formed with the receiver portion 524 and the insertion portion 526. A first piece of foam 120 (not shown) may be fixedly attached to the second locking structure 520 (e.g., ref. 122) and/or the body portion 522. While not shown in FIGS. 21-24, the insertion portion 526 may be insertable into the receiver portion 504 of the first locking structure 500 (e.g., ref. 112). The receiver portion 524 of the second locking structure 520 may be configured to engage with the third locking structure 540. In some embodiments, the receiver portion 524 may comprise a receptacle having an inner surface 525 that is angled radially outward in a distal direction (e.g., toward the insertion portion 526). In some embodiments, the insertion portion 526 may comprise a plurality of longitudinally oriented tabs 528 that may be self-biased toward an expanded position. In some embodiments, each longitudinally oriented tab of the plurality of longitudinally oriented tabs 528 may comprise an outer surface 529 that is angled radially outward in the distal direction (e.g., toward the insertion portion 526). In some embodiments, the plurality of longitudinally oriented tabs 528 may comprise a collective outer surface that is circumferentially discontinuous and is angled radially outward in the distal direction (e.g., toward the insertion portion 526). In one example, the collective outer surface may be substantially conical in shape.

In some embodiments, the plurality of longitudinally oriented tabs 528 may be resiliently deflectable radially inward toward and/or to a collapsed position by a ring member 530 movably disposed around the plurality of longitudinally oriented tabs 528 to permit the insertion portion 526 and/or the plurality of longitudinally oriented tabs 528 to be inserted into the receiver portion 504 of the first locking structure 500 (not shown). In some embodiments, the ring member 530 may be actuatable in a distal direction relative to the body portion 522, the insertion portion 526, and/or the plurality of longitudinally oriented tabs 528 by a tubular member (not shown) slidably disposable over the body portion 522 to shift the plurality of longitudinally oriented tabs 528 toward and/or to the collapsed position. With the plurality of longitudinally oriented tabs 528 in the collapsed position, the insertion portion 526 and/or the plurality of longitudinally oriented tabs 528 may be configured to be inserted into the receiver portion 504 of the first locking structure 500.

In some embodiments, a first portion (e.g., the ring member 530) of the second locking structure 520 may be movable axially relative to a second portion (e.g., the insertion portion 526 and/or the plurality of longitudinally oriented tabs 528) of the second locking structure 520 to lock the second locking structure 520 to the first locking structure 500. For example, after inserting the insertion portion 526 and/or the plurality of longitudinally oriented tabs 528 of the second locking structure 520 into the receiver portion 504 of the first locking structure 500, the tubular member may be withdrawn, thereby permitting the ring member 530 to slide proximally along the insertion portion 526 and/or the plurality of longitudinally oriented tabs 528 such that the plurality of longitudinally oriented tabs 528 may radially expand toward and/or to the expanded position within the receptacle of the first locking structure 500 to engage the collective outer surface against the inner surface 505 of the receptacle of the first locking structure 500 to actively lock the insertion portion 526 of the second locking structure 520 to the receiver portion 504 of the first locking structure 500 when the insertion portion 526 is disposed within the receptacle of the first locking structure 500.

In some embodiments, the body portion 522 may be operatively coupled to a second delivery device (not shown) and/or an elongate shaft of the second delivery device. The tubular member may be slidably disposed over and/or around the second delivery device and/or the elongate shaft of the second delivery device in order to translate and/or actuate the ring member 530 relative to the body portion 522, the insertion portion 526, and/or the plurality of longitudinally oriented tabs 528. Other configurations are also contemplated. After locking the second locking structure 520 to the first locking structure 500, the second delivery device and/or an elongate shaft of the second delivery device may be decoupled from the second locking structure 520 and removed.

Turning now more specifically to FIGS. 21-24, in some embodiments, the third locking structure 540 may comprise a body portion 542, a receiver portion 544 disposed at a proximal end of the body portion 542, and an insertion portion 546 disposed at a distal end of the body portion 542. In at least some embodiments, the body portion 542 may be monolithically formed with the receiver portion 544 and the insertion portion 546. A second piece of foam 130 (not shown) may be fixedly attached to the third locking structure 540 (e.g., ref. 132) and/or the body portion 542. The insertion portion 546 may be insertable into the receiver portion 524 of the second locking structure 520 (e.g., ref. 122). The receiver portion 544 of the third locking structure 540 may be configured to engage with the fourth locking structure (e.g., ref. 142). In some embodiments, the receiver portion 544 of the third locking structure 540 may comprise a receptacle having an inner surface 545 that is angled radially outward in a distal direction (e.g., toward the insertion portion 546). In some embodiments, the insertion portion 546 of the third locking structure 540 may comprise a plurality of longitudinally oriented tabs 548 that may be self-biased toward an expanded position. In some embodiments, each longitudinally oriented tab of the plurality of longitudinally oriented tabs 548 may comprise an outer surface 549 that is angled radially outward in the distal direction (e.g., toward the insertion portion 546). In some embodiments, the plurality of longitudinally oriented tabs 548 may comprise a collective outer surface that is circumferentially discontinuous and is angled radially outward in the distal direction (e.g., toward the insertion portion 546). In one example, the collective outer surface may be substantially conical in shape.

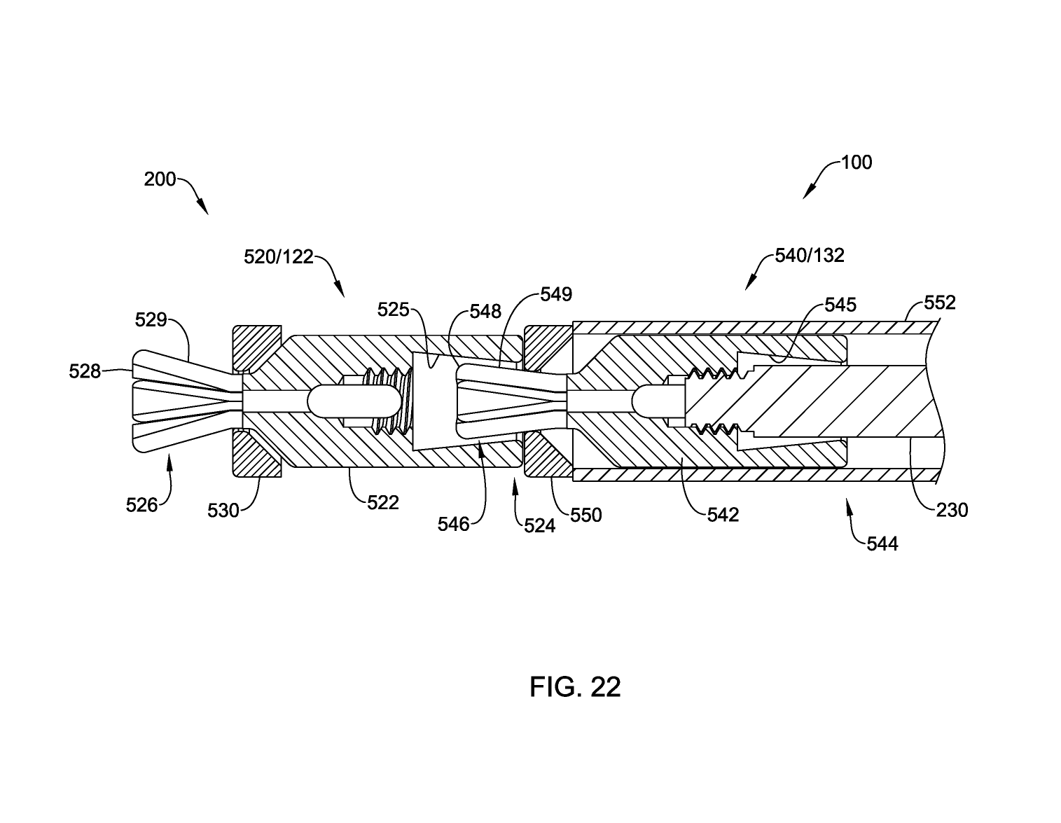

In some embodiments, the plurality of longitudinally oriented tabs 548 may be resiliently deflectable radially inward toward and/or to a collapsed position by a ring member 550 movably disposed around the plurality of longitudinally oriented tabs 548 to permit the insertion portion 546 and/or the plurality of longitudinally oriented tabs 548 to be inserted into the receiver portion 534 of the second locking structure 520. In some embodiments, the ring member 550 may be actuatable in a distal direction relative to the body portion 542, the insertion portion 546, and/or the plurality of longitudinally oriented tabs 548 by a tubular member 552 slidably disposable over the body portion 542 to shift the plurality of longitudinally oriented tabs 548 toward and/or to the collapsed position, as seen in FIG. 21. With the plurality of longitudinally oriented tabs 548 in the collapsed position, the insertion portion 546 and/or the plurality of longitudinally oriented tabs 548 may be configured to be inserted into the receiver portion 524 of the second locking structure 520, as seen in FIG. 22. In the collapsed position, an outermost radial extent of the plurality of longitudinally oriented tabs 548 may be less than an innermost radial extent of the receiver portion 544 at its proximal end.

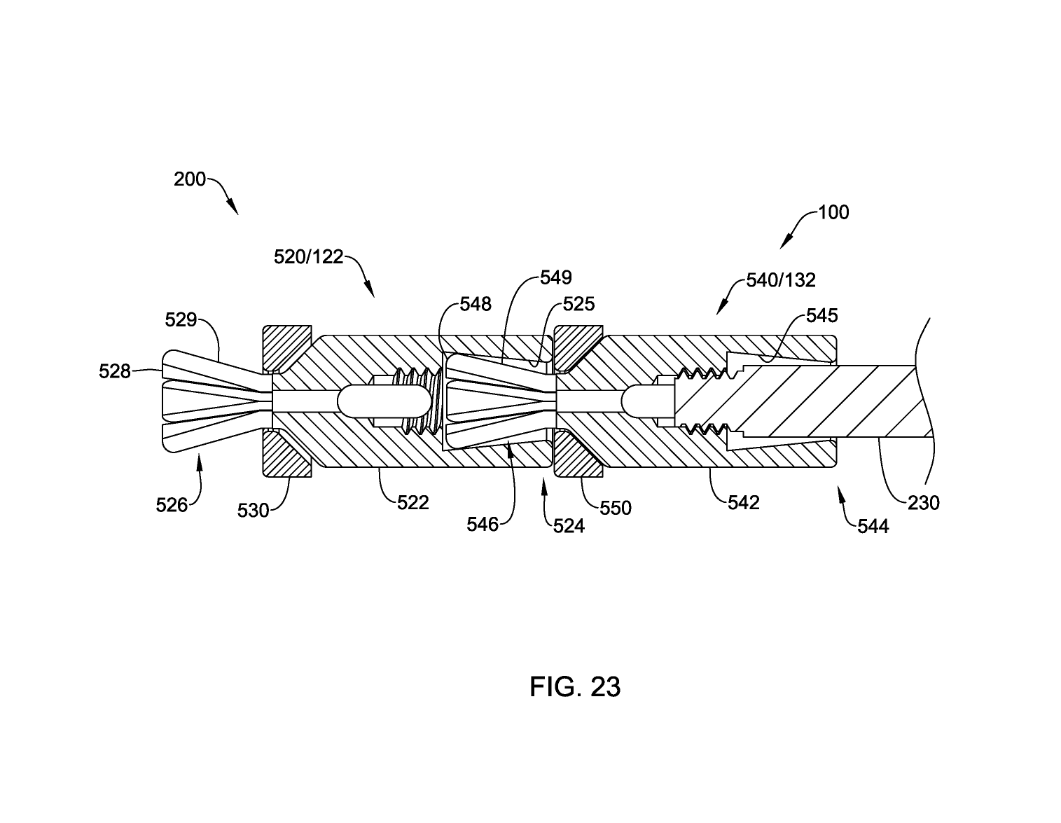

In some embodiments, a first portion (e.g., the ring member 550) of the third locking structure 540 may be movable axially relative to a second portion (e.g., the insertion portion 546 and/or the plurality of longitudinally oriented tabs 548) of the third locking structure 540 to lock the third locking structure 540 to the second locking structure 520. For example, after inserting the insertion portion 546 and/or the plurality of longitudinally oriented tabs 548 of the third locking structure 540 into the receiver portion 524 of the second locking structure 520, the tubular member 552 may be withdrawn, thereby permitting the ring member 550 to slide proximally along the insertion portion 546 and/or the plurality of longitudinally oriented tabs 548 such that the plurality of longitudinally oriented tabs 548 may radially expand toward and/or to the expanded position within the receptacle of the second locking structure 520 to engage the collective outer surface against the inner surface 525 of the receptacle of the second locking structure 520 to actively lock the insertion portion 546 of the third locking structure 540 to the receiver portion 524 of the second locking structure 520 when the insertion portion 546 is disposed within the receptacle of the second locking structure 520, as seen in FIG. 23. In the expanded position, the outermost radial extent of the plurality of longitudinally oriented tabs 548 may be greater than the innermost radial extent of the receiver portion 524 of the second locking structure 520 at its proximal end, thereby preventing the insertion portion 546 and/or the plurality of longitudinally oriented tabs 548 from being removed from the receiver portion 524 and/or the receptacle of the second locking structure 520.

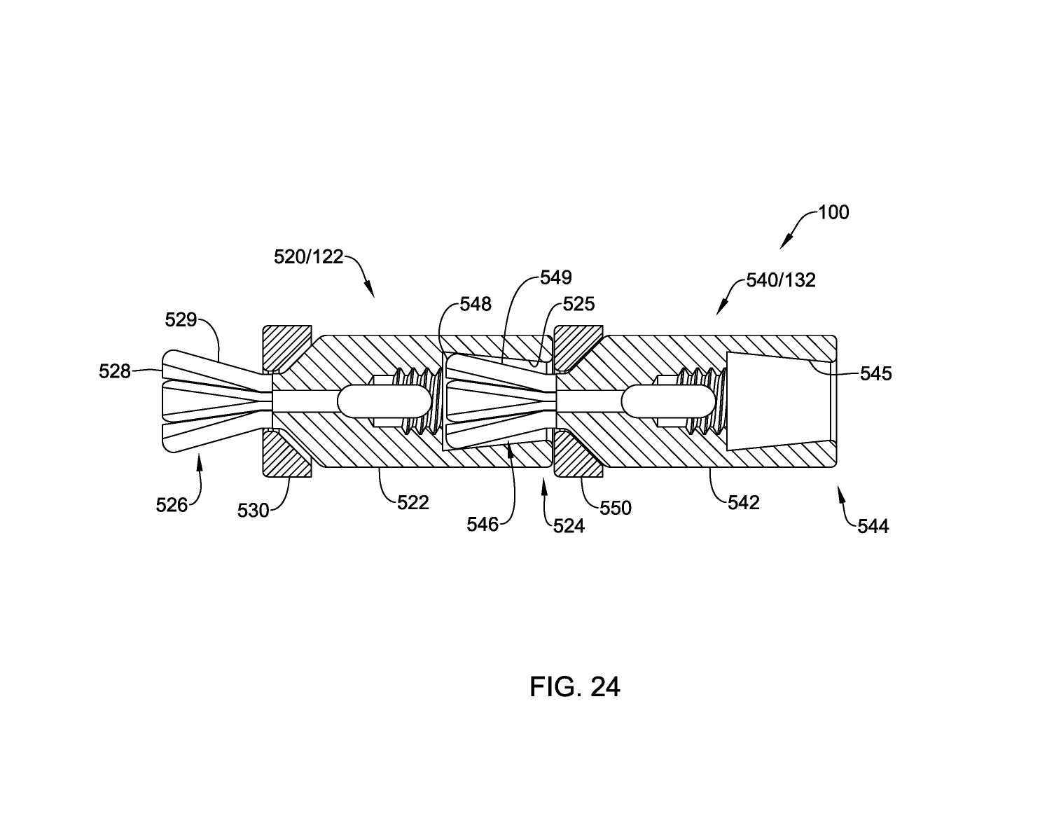

In some embodiments, the body portion 542 may be operatively coupled to the third delivery device 230 and/or an elongate shaft of the third delivery device 230. The tubular member 552 may be slidably disposed over and/or around the third delivery device 230 and/or the elongate shaft of the third delivery device 230 in order to translate and/or actuate the ring member 550 relative to the body portion 542, the insertion portion 546, and/or the plurality of longitudinally oriented tabs 548, as seen in FIGS. 21-23. Other configurations are also contemplated. After locking the third locking structure 540 to the second locking structure 520, the third delivery device 230 and/or an elongate shaft of the third delivery device 230 may be decoupled from the third locking structure 540 and removed, as seen in FIG. 24.

The materials that can be used for the various components of the system (and/or other elements disclosed herein) and the various components thereof disclosed herein may include those commonly associated with medical devices and/or systems. For simplicity purposes, the following discussion refers to the system. However, this is not intended to limit the devices and methods described herein, as the discussion may be applied to other elements, members, components, or devices disclosed herein, such as, but not limited to, the piece(s) of foam, the implant anchor element, the locking structure(s), the tubular member, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP). For example, the mixture can contain up to about 6 percent LCP.

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, the system and/or components thereof may include a fabric material. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or components thereof may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum or a Ni-Co-Cr-based alloy. The yarns may further include carbon, glass or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or components thereof may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

The foam may include any suitable material, such as a suitable polymeric material, that is capable of transitioning from an initial configuration to an expanded configuration upon being subjected to a specific temperature or temperature range and/or exposure to moisture, and provide a suitable density in the expanded configuration for use inside of the left atrial appendage to provide an occlusive benefit without negatively impacting surrounding anatomy. The foam may be a shape memory foam. Suitable transition temperatures may be, for example, at or below about 37 °C (about 98.6 °F), which allows the shape memory foam to assume an initial configuration prior to and during delivery through a delivery catheter or other delivery device, and an expanded configuration for occlusion after delivery and release within the left atrial appendage, allowing the shape memory foam to be exposed to body temperature blood within the left atrial appendage. A suitable density of the shape memory foam in the expanded configuration is a density that allows the expanded configuration to be pliable and compliant and substantially conform to the left atrial appendage anatomy to create a seal to protect against the formation and escape of blood clots while having sufficient radial force to seal the left atrial appendage but not damage or impact surrounding anatomy. In some instances, the density of the shape memory foam in the expanded configuration will be from about 10 kg/m3 (about 0.62 lb/ft3) to about 1000 kg/m3 (about 62.31 lb/ft3), including from about 10 kg/m3 to about 500 kg/m3 (about 31.2 lb/ft3) including from about 10 kg/m3 to about 200 kg/m3 (about 12.5 lb/ft3), including from about 20 kg/m3 to about 100 kg/m3 (about 6.2 lb/ft3).

Generally, the material for constructing the foam is a polymeric material that is both biocompatible and substantially biostable. In some instances, biocompatibility will include meeting or surpassing the requirements of established standards for implant materials defined in ISO 10993 and USP Class VI. Substantially biostable materials include those materials that do not resorb over the intended lifetime of the medical device (such as five years, or ten years, or longer), as well as those materials that resorb slowly such that void volume is replaced by a stable tissue-like material over a period of a few months to a year.

In some instances, the foam may include a natural and/or synthetic material. Suitable natural materials may include, for example, extracellular matrix (ECM) biopolymers such as collagen, fibronectin, hyaluronic acid and elastin, non-ECM biomaterials such as cross-linked albumin, fibrin, and inorganic bioceramics such as hydroxyapatite and tricalcium phosphate. Suitable synthetic materials may include, for example, biostable polymers such as saturated and unsaturated polyolefins including polyethylene, polyacrylics, polyacrylates, polymethacrylates, polyamides, polyimides, polyurethanes, polyureas, polyvinyl aromatics such as polystyrene, polyisobutylene copolymers and isobutylene-styrene block copolymers such as styrene-isobutylene-styrene tert-block copolymers (SIBS), polyvinylpyrolidone, polyvinyl alcohols, copolymers of vinyl monomers such as ethylene vinyl acetate (EVA), polyvinyl ethers, polyesters including polyethylene terephthalate, polyacrylamides, polyethers such as polyethylene glycol, polytetrahydrofuran and polyether sulfone, polycarbonates, silicones such as siloxane polymers, and fluoropolymers such as polyvinylidene fluoride, and mixtures and copolymers of the above.

In some instances, the foam may include a bioresorbable material such that resorption results in the formation of a biostable tissue matrix. Synthetic bioresorbable polymers may, for example, be selected from the following: (a) polyester homopolymers and copolymers such as polyglycolide (PGA; polyglycolic acid), polylactide (PLA; polylactic acid) including poly-L-lactide, poly-D-lactide and poly-D,L-lactide, poly(beta-hydroxybutyrate), polygluconate including poly-D-gluconate, poly-L-gluconate, poly-D,L-gluconate, poly(epsilon-caprolactone), poly(delta-valerolactone), poly(p-dioxanone), poly(lactide-co-glycolide) (PLGA), poly(lactide-codelta-valerolactone), poly(lactide-co-epsilon-caprolactone), poly(lactide-co-beta-malic acid), poly(beta-hydroxybutyrate-co-beta hydroxyvalerate), poly[1,3bis(p-carboxyphenoxy)propane-co-sebacic acid], and poly(sebacic acid-co-fumaric acid); (b) polycarbonate homopolymers and copolymers such as poly(trimethylene carbonate), poly(lactide-co-trimethylene carbonate) and poly(glycolide-co-trimethylene carbonate); (c) poly(ortho ester homopolymers and copolymers such as those synthesized by copolymerzation of various diketene acetals and diols; (d) polyanhydride homopolymers and copolymers such as poly(adipic anhydride), poly(suberic anhydride), poly (sebacic anhydride), poly(dodecanedioic anhydride), poly(maleic anhydride), poly[1,3-bis-(p-carboxyphenoxy)methane anhydride], and poly[alpha,omega-bis(p-carboxyphenoxy)alkane anhydride] such as poly[1,3-bis(p-carboxyphenoxy)propane anhydride] and poly[1,3-bis(p-carboxyphenoxy)hexane anhydride]; (e) polyphosphazenes such as aminated and alkoxy substituted polyphosphazenes; and (f) amino-acid-based polymers including tyrosine-based polymers such as tyrosine-based polyacrylates (e.g., copolymers of a diphenol and a diacid linked by ester bonds, with diphenols selected, for example, from ethyl, butyl, hexyl, octyl, and benzyl esters of desaminotyrosyl-tyrosine and diacids selected, for example, from succinic, glutaric, adipic, suberic, and sebacic acid), tyrosine-based polycarbonates (e.g., copolymers formed by the condensation polymerization of phosgene and a diphenol selected, for example, from ethyl, butyl, hexyl, octyl, and benzyl esters of desaminotyrosyl-tyrosine, tyrosine-based iminocarbonates, and tyrosine-, leucine- and lysine-based polyester-amides; specific examples of tyrosine-based polymers further include polymers that are comprised of a combination of desaminotyrosyl tyrosine hexyl ester, desaminotyrosyl tyrosine, and various di-acids, for example, succinic acid and adipic acid. Suitable materials include cross-linked polycarbonates and crosslinked polyethylene glycols.

In some instances, the foam may include thermoset polyurethanes that include oxidatively susceptible linkages in the soft segment, including but not limited to tertiary amines and polyethers. The shape memory foam may optionally include hydrolytically degradable soft segment components such as polycaprolactone, esters, and others. In some cases, the shape memory polymers may include non-foamed versions of the polymers described herein with respect to making the expandable foams such as shape memory foams. Example of bio-compatible shape memory polymers include polymers made from poly(ε-caprolactone) (PCL), polyurethane (PU), poly (D, L-lactide) (PDLLA), PVA, ethylene vinyl acetate copolymer, (EVA) polymer blend, polymer composites, crosslinked polymers and supramolecular networks, among others. In some instances, shape memory polymers that may be used in creating the foamable solutions described herein may include polyurethane, for example.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A device for occluding a left atrial appendage, comprising:
an implant anchor element comprising a first locking structure fixedly attached to the implant anchor element, wherein the implant anchor element is securable to tissue within the left atrial appendage;
a first piece of foam comprising a second locking structure fixedly attached to the first piece of foam, wherein the second locking structure is configured to engage the first locking structure to secure the first piece of foam to the implant anchor element; and
a second piece of foam comprising a third locking structure fixedly attached to the second piece of foam, wherein the third locking structure is configured to engage the second locking structure to secure the second piece of foam to the first piece of foam.

2. The device of claim 1, wherein the second locking structure is configured to passively lock to the first locking structure.

3. The device of claim 1, wherein the third locking structure is configured to passively lock to the second locking structure.

4. The device of claim 1, wherein the second locking structure is configured to actively lock to the first locking structure.

5. The device of claim 1, wherein the third locking structure is configured to actively lock to the second locking structure.

6. The device of claim 1, wherein a portion of the second locking structure is configured to extend into the first locking structure when the second locking structure is engaged with the first locking structure.

7. The device of claim 1, wherein a portion of the third locking structure is configured to extend into the second locking structure when the third locking structure is engaged with the second locking structure.

8. The device of claim 1, further comprising a third piece of foam comprising a fourth locking structure fixedly attached to the third piece of foam, wherein the fourth locking structure is configured to engage the third locking structure to secure the third piece of foam to the second piece of foam.

9. The device of claim 1, wherein the second locking structure is embedded within the first piece of foam.

10. The device of claim 1, wherein the third locking structure is embedded within the second piece of foam.

11. A system for occluding a left atrial appendage, comprising:
a guide sheath comprising a lumen;
a first delivery device releasably coupled to an implant anchor element comprising a first locking structure fixedly attached to the implant anchor element, wherein the first delivery device is configured to slidably pass through the lumen of the guide sheath;
a second delivery device releasably coupled to a first piece of foam comprising a second locking structure fixedly attached to the first piece of foam, wherein the second locking structure is configured to engage the first locking structure to secure the first piece of foam to the implant anchor element;
wherein the second delivery device is configured to slidably pass through the lumen of the guide sheath; and
a third delivery device releasably coupled to a second piece of foam comprising a third locking structure fixedly attached to the second piece of foam, wherein the third locking structure is configured to engage the second locking structure to secure the second piece of foam to the first piece of foam;
wherein the third delivery device is configured to slidably pass through the lumen of the guide sheath.

12. The system of claim 11, wherein the first delivery device is configured to engage the anchor implant element with tissue of the left atrial appendage.

13. The system of claim 11, wherein the second delivery device is configured to translate the first piece of foam through the lumen of the guide sheath to engage the second locking structure with the first locking structure to secure the first piece of foam to the anchor implant element.

14. The system of claim 13, wherein a first axial force applied to the second delivery device is required to translate the first piece of foam through the lumen of the guide sheath and a second axial force applied to the second delivery device is required to engage the second locking structure with the first locking structure, wherein the second axial force applied to the second delivery device is greater than the first axial force applied to the second delivery device.

15. The system of claim 11, wherein the third delivery device is configured to translate the second piece of foam through the lumen of the guide sheath to engage the third locking structure with the second locking structure to secure the second piece of foam to the first piece of foam.

16. The system of claim 15, wherein a first axial force applied to the third delivery device is required to translate the second piece of foam through the lumen of the guide sheath and a second axial force applied to the third delivery device is required to engage the third locking structure with the second locking structure, wherein the second axial force applied to the third delivery device is greater than the first axial force applied to the third delivery device.

17. The system of claim 11, wherein a first portion of the second locking structure is movable relative to a second portion of the second locking structure to lock the second locking structure to the first locking structure.

18. The system of claim 11, wherein a first portion of the third locking structure is movable relative to a second portion of the third locking structure to lock the third locking structure to the second locking structure.

19. A method of occluding a left atrial appendage, comprising:
advancing an implant anchor element releasably coupled to a first delivery device to the left atrial appendage within a lumen of a guide sheath;
securing the implant anchor element to tissue within the left atrial appendage;
removing the first delivery device from the lumen of the guide sheath;
advancing a first piece of foam releasably coupled to a second delivery device to the left atrial appendage within the lumen of the guide sheath;
engaging a first locking structure of the implant anchor element with a second locking structure of the first piece of foam to secure the first piece of foam to the implant anchor element within the left atrial appendage;
removing the second delivery device from the lumen of the guide sheath;
advancing a second piece of foam releasably coupled to a third delivery device to the left atrial appendage within the lumen of the guide sheath; and
engaging a third locking structure of the second piece of foam with the second locking structure of the first piece of foam to secure the second piece of foam to the first piece of foam within the left atrial appendage.

20. The method of claim 19, wherein engaging the first locking structure with the second locking structure comprises moving a first portion of the second locking structure relative to a second portion of the second locking structure to lock the second locking structure to the first locking structure; and
engaging the second locking structure with the third locking structure comprises moving a first portion of the third locking structure relative to a second portion of the third locking structure to lock the third locking structure to the second locking structure.
